SEQUENCE LISTING (105 sequences)

SEQ ID NO: 1 - protein, 36 aa, Homo sapiens
YPIKPEAPGEDASPEELNRYYASLRHYLNLVTRQRY

SEQ ID NO: 2 - protein, 34 aa, Homo sapiens
IKPEAPGEDASPEELNRYYASLRHYLNLVTRQRY

SEQ ID NO: 3 - protein, 15 aa, Homo sapiens
ASLRHYLNLVTRQRY

SEQ ID NO: 4 - protein, 33 aa, Homo sapiens
KPEAPGEDASPEELNRYYASLRHYLNLVTRQRY

SEQ ID NO: 5 - protein, 32 aa, Homo sapiens
PEAPGEDASPEELNRYYASLRHYLNLVTRQRY

SEQ ID NO: 6 - protein, 31 aa, Homo sapiens
EAPGEDASPEELNRYYASLRHYLNLVTRQRY

SEQ ID NO: 7 - protein, 30 aa, Homo sapiens
APGEDASPEELNRYYASLRHYLNLVTRQRY

SEQ ID NO: 8 - protein, 29 aa, Homo sapiens
PGEDASPEELNRYYASLRHYLNLVTRQRY

SEQ ID NO: 9 - protein, 28 aa, Homo sapiens
GEDASPEELNRYYASLRHYLNLVTRQRY

SEQ ID NO: 10 - protein, 27 aa, Homo sapiens
EDASPEELNRYYASLRHYLNLVTRQRY

SEQ ID NO: 11 - protein, 26 aa, Homo sapiens
DASPEELNRYYASLRHYLNLVTRQRY

SEQ ID NO: 12 - protein, 25 aa, Homo sapiens
ASPEELNRYYASLRHYLNLVTRQRY

SEQ ID NO: 13 - protein, 24 aa, Homo sapiens
SPEELNRYYASLRHYLNLVTRQRY

SEQ ID NO: 14 - protein, 23 aa, Homo sapiens
PEELNRYYASLRHYLNLVTRQRY

SEQ ID NO: 15 - protein, 22 aa, Homo sapiens
EELNRYYASLRHYLNLVTRQRY

SEQ ID NO: 16 - protein, 21 aa, Homo sapiens
ELNRYYASLRHYLNLVTRQRY

SEQ ID NO: 17 - protein, 20 aa, Homo sapiens
LNRYYASLRHYLNLVTRQRY

SEQ ID NO: 18 - protein, 19 aa, Homo sapiens
NRYYASLRHYLNLVTRQRY

SEQ ID NO: 19 - protein, 18 aa, Homo sapiens
RYYASLRHYLNLVTRQRY

SEQ ID NO: 20 - protein, 17 aa, Homo sapiens
YYASLRHYLNLVTRQRY

SEQ ID NO: 21 - protein, 16 aa, Homo sapiens
YASLRHYLNLVTRQRY

SEQ ID NO: 22 - protein, 36 aa, Homo sapiens
YPSKPDNPGEDAPAEDMARYYSALRHYINLITRQRY

SEQ ID NO: 23 - protein, 11 aa, Homo sapiens
HYINLITRQRY

SEQ ID NO: 24 - protein, 34 aa, Homo sapiens
SKPDNPGEDAPAEDMARYYSALRHYINLITRQRY

SEQ ID NO: 25 - protein, 33 aa, Homo sapiens
KPDNPGEDAPAEDMARYYSALRHYINLITRQRY

SEQ ID NO: 26 - protein, 32 aa, Homo sapiens
PDNPGEDAPAEDMARYYSALRHYINLITRQRY

SEQ ID NO: 27 - protein, 31 aa, Homo sapiens
DNPGEDAPAEDMARYYSALRHYINLITRQRY

SEQ ID NO: 28 - protein, 30 aa, Homo sapiens
NPGEDAPAEDMARYYSALRHYINLITRQRY

SEQ ID NO: 29 - protein, 29 aa, Homo sapiens
PGEDAPAEDMARYYSALRHYINLITRQRY

SEQ ID NO: 30 - protein, 28 aa, Homo sapiens
GEDAPAEDMARYYSALRHYINLITRQRY

SEQ ID NO: 31 - protein, 27 aa, Homo sapiens
EDAPAEDMARYYSALRHYINLITRQRY

SEQ ID NO: 32 - protein, 26 aa, Homo sapiens
DAPAEDMARYYSALRHYINLITRQRY

SEQ ID NO: 33 - protein, 25 aa, Homo sapiens
APAEDMARYYSALRHYINLITRQRY

SEQ ID NO: 34 - protein, 24 aa, Homo sapiens
PAEDMARYYSALRHYINLITRQRY

SEQ ID NO: 35 - protein, 23 aa, Homo sapiens
AEDMARYYSALRHYINLITRQRY

SEQ ID NO: 36 - protein, 22 aa, Homo sapiens
EDMARYYSALRHYINLITRQRY

SEQ ID NO: 37 - protein, 21 aa, Homo sapiens
DMARYYSALRHYINLITRQRY

SEQ ID NO: 38 - protein, 20 aa, Homo sapiens
MARYYSALRHYINLITRQRY

SEQ ID NO: 39 - protein, 19 aa, Homo sapiens
ARYYSALRHYINLITRQRY

SEQ ID NO: 40 - protein, 18 aa, Homo sapiens
RYYSALRHYINLITRQRY

SEQ ID NO: 41 - protein, 17 aa, Homo sapiens
YYSALRHYINLITRQRY

SEQ ID NO: 42 - protein, 16 aa, Homo sapiens
YSALRHYINLITRQRY

SEQ ID NO: 43 - protein, 15 aa, Homo sapiens
SALRHYINLITRQRY

SEQ ID NO: 44 - protein, 14 aa, Homo sapiens
ALRHYINLITRQRY

SEQ ID NO: 45 - protein, 13 aa, Homo sapiens
LRHYINLITRQRY

SEQ ID NO: 46 - protein, 12 aa, Homo sapiens
RHYINLITRQRY

SEQ ID NO: 47 - protein, 36 aa, Homo sapiens
ASLEPEYPGDNATPEQMAQYAAELRRYINMLTRPRY

SEQ ID NO: 48 - protein, 11 aa, Homo sapiens
RYINMLTRPRY

SEQ ID NO: 49 - protein, 34 aa, Homo sapiens
LEPEYPGDNATPEQMAQYAAELRRYINMLTRPRY

SEQ ID NO: 50 - protein, 33 aa, Homo sapiens
EPEYPGDNATPEQMAQYAAELRRYINMLTRPRY

SEQ ID NO: 51 - protein, 32 aa, Homo sapiens
PEYPGDNATPEQMAQYAAELRRYINMLTRPRY

SEQ ID NO: 52 - protein, 31 aa, Homo sapiens
EYPGDNATPEQMAQYAAELRRYINMLTRPRY

SEQ ID NO: 53 - protein, 30 aa, Homo sapiens
YPGDNATPEQMAQYAAELRRYINMLTRPRY

SEQ ID NO: 54 - protein, 29 aa, Homo sapiens
PGDNATPEQMAQYAAELRRYINMLTRPRY

SEQ ID NO: 55 - protein, 28 aa, Homo sapiens
GDNATPEQMAQYAAELRRYINMLTRPRY

SEQ ID NO: 56 - protein, 27 aa, Homo sapiens
DNATPEQMAQYAAELRRYINMLTRPRY

SEQ ID NO: 57 - protein, 26 aa, Homo sapiens
NATPEQMAQYAAELRRYINMLTRPRY

SEQ ID NO: 58 - protein, 25 aa, Homo sapiens
ATPEQMAQYAAELRRYINMLTRPRY

SEQ ID NO: 59 - protein, 24 aa, Homo sapiens
TPEQMAQYAAELRRYINMLTRPRY

SEQ ID NO: 60 - protein, 23 aa, Homo sapiens
PEQMAQYAAELRRYINMLTRPRY

SEQ ID NO: 61 - protein, 22 aa, Homo sapiens
EQMAQYAAELRRYINMLTRPRY

SEQ ID NO: 62 - protein, 21 aa, Homo sapiens
QMAQYAAELRRYINMLTRPRY

SEQ ID NO: 63 - protein, 20 aa, Homo sapiens
MAQYAAELRRYINMLTRPRY

SEQ ID NO: 64 - protein, 19 aa, Homo sapiens
AQYAAELRRYINMLTRPRY

SEQ ID NO: 65 - protein, 18 aa, Homo sapiens
QYAAELRRYINMLTRPRY

SEQ ID NO: 66 - protein, 17 aa, Homo sapiens
YAAELRRYINMLTRPRY

SEQ ID NO: 67 - protein, 16 aa, Homo sapiens
AAELRRYINMLTRPRY

SEQ ID NO: 68 - protein, 15 aa, Homo sapiens
AELRRYINMLTRPRY

SEQ ID NO: 69 - protein, 14 aa, Homo sapiens
ELRRYINMLTRPRY

SEQ ID NO: 70 - protein, 13 aa, Homo sapiens
LRRYINMLTRPRY

SEQ ID NO: 71 - protein, 12 aa, Homo sapiens
RRYINMLTRPRY

SEQ ID NO: 72 - protein, 36 aa, Rat
YPAKPEAPGEDASPEELSRYYASLRHYLNLVTRQRY

SEQ ID NO: 73 - protein, 36 aa, Pig; identical to SEQ ID NO: 72

SEQ ID NO: 74 - protein, 36 aa, Guinea pig
YPSKPEAPGSDASPEELARYYASLRHYLNLVTRQRY

SEQ ID NO: 75 - protein, 36 aa, Rat; identical to SEQ ID NO: 22

SEQ ID NO: 76 - protein, 36 aa, Rabbit; identical to SEQ ID NO: 22

SEQ ID NO: 77 - protein, 36 aa, Dog; identical to SEQ ID NO: 22

SEQ ID NO: 78 - protein, 36 aa, Pig
YPSKPDNPGEDAPAEDLARYYSALRHYINLITRQRY

SEQ ID NO: 79 - protein, 36 aa, Cow; identical to SEQ ID NO: 78

SEQ ID NO: 80 - protein, 36 aa, Sheep
YPSKPDNPGDDAPAEDLARYYSALRHYINLITRQRY

SEQ ID NO: 81 - protein, 36 aa, Guinea pig; identical to SEQ ID NO: 22

SEQ ID NO: 82 - protein, 36 aa, Sheep
APLEPVYPGDNATPEQMAQYAADLRRYINMLTRPRY

SEQ ID NO: 83 - protein, 36 aa, Pig
APLEPVYPGDDATPEQMAQYAAELRRYINMLTRPRY

SEQ ID NO: 84 - protein, 36 aa, Dog; identical to SEQ ID NO: 83

SEQ ID NO: 85 - protein, 36 aa, Cat
APLEPVYPGDNATPEQMAQYAAELRRYINMLTRPRY

SEQ ID NO: 86 - protein, 36 aa, Cow
APLEPEYPGDDATPEQMAQYAAELRRYINMLTRPRY

SEQ ID NO: 87 - protein, 36 aa, Rat
APLEPMYPGDYATHEQRAQYETQLRRYINTLTRPRY

SEQ ID NO: 88 - protein, 36 aa, mouse
APLEPMYPGDYATPEQMAQYETQLRRYINTLTRPRY

SEQ ID NO: 89 - protein, 37 aa, Guinea pig
APLEPVYPGDNATPEQQMAQYAAEMRRYINMLTRPRY

SEQ ID NO: 90 - protein, 22 aa, Homo sapiens
DELNRYYASLRHYLNLVTRQRY

SEQ ID NO: 91 - protein, 24 aa, Homo sapiens
TPEELNRYYASLRHYLNLVTRQRY

SEQ ID NO: 92 - protein, 25 aa, Homo sapiens
VSPEELNRYYASLRHYLNLVTRQRY

SEQ ID NO: 93 - protein, 26 aa, Homo sapiens
EASPEELNRYYASLRHYLNLVTRQRY

SEQ ID NO: 94 - protein, 27 aa, Homo sapiens
DDASPEELNRYYASLRHYLNLVTRQRY

SEQ ID NO: 95 - protein, 30 aa, Homo sapiens
VPGEDASPEELNRYYASLRHYLNLVTRQRY

SEQ ID NO: 96 - protein, 31 aa, Homo sapiens
DAPGEDASPEELNRYYASLRHYLNLVTRQRY

SEQ ID NO: 97 - protein, 33 aa, Homo sapiens
QPEAPGEDASPEELNRYYASLRHYLNLVTRQRY

SEQ ID NO: 98 - protein, 33 aa, Homo sapiens
RPEAPGEDASPEELNRYYASLRHYLNLVTRQRY

SEQ ID NO: 99 - protein, 33 aa, Homo sapiens
NPEAPGEDASPEELNRYYASLRHYLNLVTRQRY

SEQ ID NO: 100 - protein, 34 aa, Homo sapiens
VKPEAPGEDASPEELNRYYASLRHYLNLVTRQRY

SEQ ID NO: 101 - protein, 34 aa, Homo sapiens
LKPEAPGEDASPEELNRYYASLRHYLNLVTRQRY

SEQ ID NO: 102 - protein, 27 aa, Homo sapiens
DDASPDELNRYYASLRHYLNLVTRQRY

SEQ ID NO: 103 - protein, 31 aa, Homo sapiens
DAPGEDATPEELNRYYASLRHYLNLVTRQRY

SEQ ID NO: 104 - protein, 33 aa, Homo sapiens
NPEAPGEDASPDELNRYYASLRHYLNLVTRQRY

SEQ ID NO: 105 - protein, 34 aa, Homo sapeins
LKPEAPGDDASPEELNRYYASLRHYLNLVTRQRY

(19) United States
(12) Patent Application Publication
(10) Pub. No.: US 2007/0213270 A1
(43) Pub. Date: Sep. 13, 2007

(54) PEPTIDE YY FORMULATIONS HAVING INCREASED STABILITY AND RESISTANCE TO MICROBIAL AGENTS

(76) Inventors: Henry R. Costantino, Woodinville, WA (US); Mary S. Kleppe, Snohomish, WA (US); Annemarie Stoudt Cohen, Kirkland, WA (US)

Correspondence Address: NASTECH PHARMACEUTICAL COMPANY INC, 3830 MONTE VILLA PARKWAY, BOTHELL, WA, 98021-7266, US

(21) Appl. No.: 11/570,223

(22) PCT Filed: Jun. 16, 2005
(86) PCT No.: PCT/US05/21377

§ 371 Date: Dec. 7, 2006

Related U.S. Application Data
(60) Provisional application No. 60/580,329, filed on Jun. 16, 2004.
(60) Provisional application No. 60/580,310, filed on Jun. 16, 2004.

(51) Int. Cl. A61K 38/22; A61K 31/724

(52) U.S. Cl. 514/12; 514/58

(57) ABSTRACT

Pharmaceutical compositions and methods are described comprising at least one Y2 receptor-binding peptide, such as peptide YY (PYY), Neuropeptide Y (NPY) or Pancreatic Peptide (PP) wherein the formulations have increased resistance to microbial contamination and is comprised of a Y2 receptor-binding peptide, water, a cyclodextrin and sodium benzoate.

This application claims priority under 35 U.S.C. §371(c) of co-pending PCT Application No. PCT/US2005/021377 filed Jun. 16, 2005, which claims priority to U.S. Provisional Application No. 60/580,329, filed Jun. 16, 2004 and U.S. Provisional Application No. 60/580,310, filed Jun. 16, 2004; the entire contents of these applications are incorporated herein by reference.

Obesity and its associated disorders are common and very serious public health problems in the United States and throughout the world. Upper body obesity is the strongest risk factor known for type-2 diabetes mellitus, and is a strong risk factor for cardiovascular disease. Obesity is a recognized risk factor for hypertension, arteriosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. It reduces life-span and carries a serious risk of co-morbidities above, as well disorders such as infections, varicose veins, acanthosis nigricans, eczema, exercise intolerance, insulin resistance, hypertension hypercholesterolemia, cholelithiasis, orthopedic injury, and thromboembolic disease. for the group of conditions called insulin resistance syndrome, or “Syndrome X.”

It has been shown that certain peptides that bind to the Y2 receptor when administered peripherally to a mammal induce weight loss. The Y2 receptor-binding peptides are neuropeptides that bind to the Y2 receptor. Neuropeptides are small peptides originating from large precursor proteins synthesized by peptidergic neurons and endocrine/paracrine cells. Often the precursors contain multiple biologically active peptides. There is great diversity of neuropeptides in the brain caused by alternative splicing of primary gene transcripts and differential precursor processing. The neuropeptide receptors serve to discriminate between ligands and to activate the appropriate signals. These Y2 receptor-binding peptides belong to a family of peptides including peptide YY (PYY), neuropeptide Y (NPY) and pancreatic peptide (PP).

NPY is a 36-amino acid peptide and is the most abundant neuropeptide to be identified in mammalian brain. NPY is an important regulator in both the central and peripheral nervous systems and influences a diverse range of physiological parameters, including effects on psychomotor activity, food intake, central endocrine secretion, and vasoactivity in the cardiovascular system. High concentrations of NPY are found in the sympathetic nerves supplying the coronary, cerebral, and renal vasculature and have contributed to vasoconstriction. NPY binding sites have been identified in a variety of tissues, including spleen, intestinal membranes, brain, aortic smooth muscle, kidney, testis, and placenta.

Neuropeptide Y (NPY) receptor pharmacology is currently defined by structure activity relationships within the pancreatic polypeptide family. This family includes NPY, which is synthesized primarily in neurons; PYY, which is synthesized primarily by endocrine cells in the gut; and PP, which is synthesized primarily by endocrine cells in the pancreas. These approximately 36 amino acid peptides have a compact helical structure involving a “PP-fold” in the middle of the peptide. Specific features include a polyproline helix in residues 1 through 8, a β-turn in residues 9 through 14, an α-helix in residues 15 through 30, an outward-projecting C-terminus in residues 30 through 36, and a carboxyl terminal amide, which appears to be critical for biological activity. The peptides have been used to define at least five receptor subtypes known as Y1, Y2, Y3, Y4 and Y5. Y1 receptor recognition by NPY involves both N- and C-terminal regions of the peptide; exchange of Gln³⁴ with Pro³⁴ is fairly well tolerated. Y2 receptor recognition by NPY depends primarily upon the four C-terminal residues of the peptide (Arg³³-Gln³⁴-Arg³-Tyr36NH₂) preceded by an amphipathic an α-helix; exchange of Gln³⁴ with Pro³⁴ is not well tolerated. One of the key pharmacological features which distinguish Y1 and Y2 is the fact that the Y2 receptor (and not the Y1 receptor) has high affinity for the NPY peptide carboxyl-terminal fragment NPY-(13-36) and the PYY fragment PYY(22-36).

It has been shown that a 36 amino acid peptide called Peptide YY(1-36) [PYY(1-36)] [YPIKPEAPGEDASPEELNRYYASLRHYLNLVTRQRY, SEQ ID NO.: 1]. when administered peripherally by injection to an individual produces weight loss and thus can be used as a drug to treat obesity and related diseases, Morley, J. Neuropsychobiology 21:22-30 (1989). It was later found that to produce this effect PYY bound to a Y2 receptor, and the binding of a Y2 agonist to the Y2 receptor caused a decrease in the ingestion of carbohydrate, protein and meal size, Leibowitz, S. F. et al. Peptides, 12: 1251-1260 (1991). An alternate molecular form of PYY is PYY(3-36) IKPEAPGEDASPEELNRYYASLRHYLNLVTRQRY [SEQ ID NO.: 2], Eberlein, Eysselein et al. Peptides 10: 797-803, 1989). This fragment constitutes approximately 40% of total PYY-like immunoreactivity in human and canine intestinal extracts and about 36% of total plasma PYY immunoreactivity in a fasting state to slightly over 50% following a meal. It is apparently a dipeptidyl peptidase-IV (DPP4) cleavage product of PYY. PYY3-36 is reportedly a selective ligand at the Y2 and Y5 receptors, which appear pharmacologically unique in preferring N-terminally truncated (i.e. C-terminal fragments of) NPY analogs. It has also been shown that a PYY fragment having only residues 22-36 will still bind to the Y2 receptor. However, if any of the carboxyl terminus of the peptide is cleaved, the peptide looses its ability to bind to the Y2 receptor. Hence a PYY agonist is a peptide, which has a partial sequence of full-length PYY and is able to bind to a Y2 receptor in the arcuate nucleus of the hypothalamus. Hereinafter the term PYY refers to full-length PYY and any fragment of PYY that binds to a Y2 receptor.

It is known that PYY and PYY3-36 can be administered by intravenous infusion or injection to treat life-threatening hypotension as encountered in shock, especially that caused by endotoxins (U.S. Pat. No. 4,839,343), to inhibit proliferation of pancreatic tumors in mammals by perfusion, parenteral, intravenous, or subcutaneous administration, and by implantation (U.S. Pat. No. 5,574,010) and to treat obesity (Morley, J. Neuropsychobiology 21:22-30 (1989) and US Patent Application 20020141985). It is also claimed that PYY can be administered by parenteral, oral, nasal, rectal and topical routes to domesticated animals or humans in an amount effective to increase weight gain of said subject by enhancing gastrointestinal absorption of a sodium-dependent cotransported nutrient (U.S. Pat. No. 5,912,227). However, for the treatment of obesity and related diseases, including diabetes, the mode of administration has been limited to intravenous IV infusion with no effective formulations optimized for alternative administration of PYY3-36. None of these prior art teachings provide formulations that contain PYY or PYY(3-36) combined with excipients designed to enhance mucosal (i.e., nasal, buccal, oral) delivery nor do they teach the value of endotoxin-free Y2-receptor binding peptide formulations for non-infused administration. Thus, there is a need to develop formulations and methods for administering PYY3-36.

### SUMMARY OF THE INVENTION

One aspect of the invention is an aqueous Y2 receptor-binding peptide formulation suitable for transmucosal administration, comprising a Y2 receptor-binding peptide, a cyclodextrin and an effective amount of an anti-microbial preservative. The cyclodextrin may be α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin or a polymeric cyclodextrin. Most preferably, the cyclodextrin is methyl-β-cyclodextrin. Most preferably, the preservative is sodium benzoate. Sodium benzoate may be used at a concentration greater than about 0.05%, preferably more than about 0.10%, more preferably greater than about 0.25%, and most preferably greater than about 0.50%. The formulation may have a pH between 3.0 and 5.0, most preferably between 3.5 and 4.5.

Another embodiment of the invention is the above formulation in which the preservative-effectiveness meets United State Pharmacopoeia for Category 2 criteria, more preferably, also meeting European Pharmacopoeia Category A criteria, most preferably meeting European Pharmacopoeia Category B criteria. Another embodiment is the formulation in which the preservative effectively causes at least a 1 log reduction for a yeast and a mold at day 14, preferably, C. albicans and A. niger species, respectively. The Y2 receptor-binding peptide formulation of claim 2, wherein the preservative effectively causes at least a 1 log reduction for a yeast and a mold at day 14, most preferably, effectively causes at least a 2 log reduction for C. albicans and A. niger species.

Another embodiment of the invention is the above formulation in which the Y2 receptor-binding peptide is PYY, PYY (3-36) or an analogue of PYY or an analog of PYY (3-36). Preferably, the Y2 receptor-binding peptide is a PYY peptide comprised of an amino acid sequence selected from the group consisting of SEQ ID NOs: 1-21, SEQ ID NOs: 72, 73 and 74, and SEQ ID NOs: 90-105. More preferably the PYY peptide is a PYY(3-36) peptide. Most preferably, the PYY(3-36) peptide is comprised of an amino acid sequence selected from the group consisting of SEQ ID NOs: 2, 3 and SEQ ID NOs: 90-105.

Another embodiment of the invention is an aqueous Y2 receptor-binding peptide formulation suitable for transmucosal administration, comprising a Y2 receptor-binding peptide and a cyclodextrin, in which the formulation has been prepared aseptically. The cyclodextrin may be selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin. Most preferably the cyclodextrin is methyl-β-cyclodextrin.

Another aspect of the invention is a method for treating obesity in a human, comprised of intranasally administering to the human a therapeutically effective amount of an aqueous formulation, in which the formulation comprises a Y2 receptor-binding peptide, a cyclodextrin and sodium benzoate. This method may induce weight-loss or satiety. The cyclodextrin may be selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin. The cyclodextrin preferably is methyl-β-cyclodextrin.

One embodiment of the invention is the method in which the preservative preferably is sodium benzoate. The sodium benzoate may be use at a concentration greater than about 0.25%, most preferably greater than about 0.50%. The method may involve a formulation with a pH between 3.0 and 5.0, preferably between 3.5 and 4.5.

Another aspect of the invention is the method, above, for treating a mammal for obesity of claim 25, in which the Y2 receptor-binding peptide is PYY or PYY (3-36) or an analogue of PYY or an analog of PYY (3-36). Preferably, the Y2 receptor-binding peptide is a PYY peptide comprised of an amino acid sequence selected from the group consisting of SEQ ID NOs: 1-21, SEQ ID NOs: 72, 73 and 74, and SEQ ID NOs: 90-105. More preferably, the PYY peptide is a PYY(3-36) peptide. Most preferably, the PYY(3-36) peptide is comprised of an amino acid sequence selected from the group consisting of SEQ ID NOs: 2, 3 and SEQ ID NOs: 90-105.

Another aspect of the invention is a method for treating obesity in a human, comprised of intranasally administering to the human a therapeutically effective amount of an aqueous formulation, in which the formulation comprises a Y2 receptor-binding peptide and a cyclodextrin, and wherein such formulation has been prepared aseptically. Preferably the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin. Most preferably, the cyclodextrin is methyl-β-cyclodextrin.

Another aspect of the invention is an aqueous Y2 receptor-binding peptide formulation suitable for transmucosal administration, comprising a Y2 receptor-binding peptide, a cyclodextrin, and having a pH between 3.0 and 6.0. One embodiment is a formulation having an osmolality between about 50 and about 300 mOsm, preferably between about 150 and about 275 mOsm, most preferably between about 180 and about 260 mOsm. Another embodiment is the formulation that is free of sugars. Another embodiment is a formulation in which the osmolarity of the formulation is adjusted using an inorganic salt, preferably sodium chloride.

Another embodiment of the invention is the formulation of the previous paragraph in which the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin. Most preferably, the cyclodextrin is methyl-β-cyclodextrin. Another embodiment is the formulation in which the pH is between about 3.5 and about 5.5, preferably between about 3.5 and about 5.0, more preferably between about 3.7 and about 4.7.

Another embodiment of the invention is the formulation of the previous paragraph in which the pH of the formulation is controlled by a buffer salt, and the buffer salt has a net single ionogenic moiety with a pKₐ within two pH units of the pH of the formulation, preferably a pKₐ within one pH unit of the pH of the formulation. The buffer salt may be selected from the list consisting of glutamate, acetate, glycine, histidine, arginine, lysine, methionine, lactate, formate, and glycolate.

Another embodiment of the invention is the formulation of the previous paragraph in which the Y2 receptor-binding peptide is PYY or an analogue of PYY. A preferred embodiment is a PYY peptide comprised of an amino acid sequence selected from the group consisting of SEQ ID NOs: 1-21, SEQ ID NOs: 72, 73 and 74, and SEQ ID NOs: 90-105. A more preferred embodiment is a formulation in which the PYY peptide is a PYY(3-36) peptide. Most preferably, the PYY(3-36) peptide is comprised of an amino acid sequence selected from the group consisting of SEQ ID NOs: 2, 3 and SEQ ID NOs: 90-105.

Another aspect of the invention is a method for treating obesity in a mammal comprising intranasally administering to the mammal a therapeutically effective amount of an aqueous formulation comprised a Y2 receptor-binding peptide in which the formulation is comprised of a cyclodextrin, has a pH between 3.0 and 6.0 and an osmolarity between about 50 mOsm and about 300 mOsm. Another embodiment is the method for treating obesity, by inducing weight-loss or inducing satiety. One embodiment of the invention is the method, in which the osmolarity of the formulation is between about 150 and about 275 mOsm, preferably between about 180 and about 260 mOsm. Another aspect of the invention is the method for treating obesity in which the formulation is free of sugars. Another embodiment is the method of treatment in the osmolarity of the formulation is adjusted using an inorganic salt, preferably sodium chloride.

Another embodiment of the invention is the method of treatment of the previous paragraph in which the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin. Most preferably, the cyclodextrin is methyl-β-cyclodextrin. Another embodiment is the method of treatment in which the formulation has a pH between about 3.5 and about 5.5, preferably between about 3.5 and about 5.0, more preferably between about 3.7 and about 4.7.

Another embodiment of the invention is the method of treatment of the previous paragraph in which the pH of the formulation is controlled by a buffer salt, and the buffer salt has a net single ionogenic moiety with a pKₐ within two pH units of the pH of the formulation, preferably a pKₐ within one pH unit of the pH of the formulation. The buffer salt may be selected from the list consisting of glutamate, acetate, glycine, histidine, arginine, lysine, methionine, lactate, formate, and glycolate.

Another embodiment of the invention is the method of treatment of the previous paragraph in which the Y2 receptor-binding peptide is PYY or an analogue of PYY. A preferred embodiment is a PYY peptide comprised of an amino acid sequence selected from the group consisting of SEQ ID NOs: 1-21, SEQ ID NOs: 72, 73 and 74, and SEQ ID NOs: 90-105. A more preferred embodiment is a formulation in which the PYY peptide is a PYY(3-36) peptide. Most preferably, the PYY(3-36) peptide is comprised of an amino acid sequence selected from the group consisting of SEQ ID NOs: 2, 3 and SEQ ID NOs: 90-105.

Another aspect of the invention is an aqueous Y2 receptor-binding peptide formulation suitable for transmucosal administration, comprising a Y2 receptor-binding peptide, a cyclodextrin and has a pH between 3.0 and 6.0.

Another aspect of the invention is a method for treating obesity in a mammal comprising intranasally administering to said mammal a therapeutically effective amount of an aqueous formulation comprised a Y2 receptor-binding peptide wherein the formulation is comprised of a cyclodextrin and has a pH between 3.0 and 6.0 and an osmolarity between about 50 mOsm and about 300 mOsm.

### BRIEF DESCRIPTION OF THE DRAWINGS

FIG. 1: Effect of Osmolarity on Stability Towards Thermal Stress for PYY₃₋₃₆ in the Presence of Me-b-CD, DDPC, EDTA, Citrate Buffer, Chlorobutanol and either Sodium Chloride or Lactose/Sorbitol as Tonicifier.

FIG. 2: Comparative Stability Towards Thermal Stress for PYY₃₋₃₆ in the Presence of Me-b-CD, DDPC, EDTA, Citrate Buffer, Lactose, Sorbitol and either Chlorobutanol or Sodium Benzoate.

FIG. 3: Effect of Stability Towards Thermal Stress for PYY₃₋₃₆ in the Presence of Me-b-CD, DDPC, EDTA, Citrate Buffer, Chlorobutanol and either Lactose, Sorbitol and/or Sodium Chloride.

FIG. 4: (A) Effect of pH on Stability Towards Thermal Stress for PYY₃₋₃₆ in the Presence of Me-b-CD, DDPC, EDTA, Citrate Buffer, Chlorobutanol and Sodium Chloride as the Tonicifer; (B) Effect of pH upon PYY₃₋₃₆ stability, 40° C. storage.

FIG. 5: Effects of Preservative on Stability Towards Thermal Stress for PYY₃₋₃₆ in the Presence of Me-b-CD, DDPC, EDTA, Citrate Buffer, Lactose and Sorbitol.

FIG. 6: (A) Peptide Recovery vs. Time, 25° C. storage; (B): Peptide Recovery vs. Time, 40° C. storage; and (C): Peptide Recovery vs. Time, 50° C. storage.

FIG. 7: (A) Peptide recovery vs. time, citrate buffer-based formulations, 40° C. storage; (B) Peptide recovery vs. time, acetate buffer-based formulations, 40° C. storage; (C) Peptide recovery vs. time, glutamate buffer-based formulations, 40° C. storage; and (D) Peptide recovery vs. time, unbuffered formulations, 40° C. storage.

FIG. 8: (A) Peptide recovery over ten days' of thermal and actuation stress, citrate-based formulations; (B) Peptide recovery over ten days' of thermal and actuation stress, acetate-based formulations; (C) Peptide recovery over ten days' of thermal and actuation stress, glutamate-based formulations; and (D) Peptide recovery over ten days' of thermal and actuation stress, unbuffered formulations.

### DETAILED DESCRIPTION OF THE INVENTION

In exemplary embodiments, the enhanced delivery methods and compositions of the present invention provide for therapeutically effective mucosal delivery of the Y2 receptor-binding peptide agonist for prevention or treatment of obesity and eating disorders in mammalian subjects. In one aspect of the invention, pharmaceutical formulations suitable for intranasal administration are provided that comprise a therapeutically effective amount of a Y2 receptor-binding peptide and one or more intranasal delivery-enhancing agents as described herein, which formulations are effective in a nasal mucosal delivery method of the invention to prevent the onset or progression of obesity or eating disorders in a mammalian subject. Nasal mucosal delivery of a therapeutically effective amount of a Y2 receptor-binding peptide agonist and one or more intranasal delivery-enhancing agents yields elevated therapeutic levels of the Y2 receptor-binding peptide agonist in the subject and inhibits food intake in the mammalian subject, reducing symptoms of obesity or an eating disorder.

The enhanced delivery methods and compositions of the present invention provide for therapeutically effective mucosal delivery of a Y2 receptor-binding peptide for prevention or treatment of a variety of diseases and conditions in mammalian subjects. Y2 receptor-binding peptide can be administered via a variety of mucosal routes, for example by contacting the Y2 receptor-binding peptide to a nasal mucosal epithelium, a bronchial or pulmonary mucosal epithelium, the oral buccal surface or the oral and small intestinal mucosal surface. In exemplary embodiments, the methods and compositions are directed to or formulated for intranasal delivery (e.g., nasal mucosal delivery or intranasal mucosal delivery).

In one aspect of the invention, pharmaceutical formulations suitable for intranasal administration are provided that comprise a therapeutically effective amount of a Y2 receptor-binding peptide agonist and one or more intranasal delivery-enhancing agents as described herein, which formulations are effective in a nasal mucosal delivery method of the invention to prevent the onset or progression of obesity, diabetes, cancer, or malnutrition or wasting related to cancer in a mammalian subject, or to alleviate one or more clinically well-recognized symptoms of obesity, as well as treating Alzheimer's disease, colon carcinoma, colon adenocarcinoma, pancreatic carcinoma, pancreatic adenocarcinoma, breast carcinoma.

In another aspect of this invention, it was surprisingly found that the use of endotoxin-free Y2 receptor binding peptides, for example PYY(3-36), produced increased mucosal delivery compared to peptides in which endotoxin is not removed. The use of endotoxin-free Y2 receptor peptides in pharmaceutical formulations is thus enabled for administration by non-infusion routes, including mucosal delivery, nasal, oral, pulmonary, vaginal, rectal and the like.

The foregoing mucosal Y2 receptor-binding peptide formulations and preparative and delivery methods of the invention provide improved mucosal delivery of a Y2 receptor-binding peptide to mammalian subjects. These compositions and methods can involve combinatorial formulation or coordinate administration of one or more Y2 receptor-binding peptides with one or more mucosal delivery-enhancing agents. Among the mucosal delivery-enhancing agents to be selected from to achieve these formulations and methods are (A) solubilization agents; (B) charge modifying agents; (C) pH control agents; (D) degradative enzyme inhibitors; (E) mucolytic or mucus clearing agents; (F) ciliostatic agents; (G) membrane penetration-enhancing agents (e.g., (i) a surfactant, (ii) a bile salt, (iii) a phospholipid or fatty acid additive, mixed micelle, liposome, or carrier, (iv) an alcohol, (v) an enamine, (iv) an NO donor compound, (vii) a long-chain amphipathic molecule (viii) a small hydrophobic penetration enhancer; (ix) sodium or a salicylic acid derivative; (x) a glycerol ester of acetoacetic acid (xi) a cyclodextrin or beta-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, (xviii) an inhibitor of cholesterol synthesis; or (xiv) any combination of the membrane penetration enhancing agents of (i)-(xviii)); (H) modulatory agents of epithelial junction physiology, such as nitric oxide (NO) stimulators, chitosan, and chitosan derivatives; (I) vasodilator agents; (J) selective transport-enhancing agents; and (K) stabilizing delivery vehicles, carriers, supports or complex-forming species with which the Y2 receptor-binding peptide (s) is/are effectively combined, associated, contained, encapsulated or bound to stabilize the active agent for enhanced mucosal delivery.

In various embodiments of the invention, a Y2 receptor-binding peptide is combined with one, two, three, four or more of the mucosal delivery-enhancing agents recited in (A)-(K), above. These mucosal delivery-enhancing agents may be admixed, alone or together, with the Y2 receptor-binding peptide, or otherwise combined therewith in a pharmaceutically acceptable formulation or delivery vehicle. Formulation of a Y2 receptor-binding peptide with one or more of the mucosal delivery-enhancing agents according to the teachings herein (optionally including any combination of two or more mucosal delivery-enhancing agents selected from (A)-(K) above) provides for increased bioavailability of the Y2 receptor-binding peptide following delivery thereof to a mucosal surface of a mammalian subject.

Thus, the present invention is a method for suppressing appetite, promoting weight loss, decreasing food intake, or treating obesity and/or diabetes in a mammal comprising transmucosally administering a formulation comprised of a Y2 receptor-binding peptide, such that when at 50 μg of the Y2 receptor is administered transmucosally to the mammal the concentration of the Y2 receptor-binding peptide in the plasma of the mammal increases by at least 5 pmol, preferably at least 10 pmol per liter of plasma. Examples of such formulations are described above.

The present invention further provides for the use of a Y2 receptor-binding peptide for the production of medicament for the transmucosal, administration of a Y2 receptor-binding peptide for suppressing appetite, promoting weight loss, decreasing food intake, or treating obesity in a mammal such that when about 50 μg of the Y2 receptor is administered transmucosally to the mammal the concentration of the Y2 receptor-binding peptide in the plasma of the mammal increases by at least 5 pmol per liter of plasma. When 100 μg of the Y2 receptor-binding peptide is administered intranasally to the mammal, the concentration of the Y2 receptor agonist increases by at least 20 pmol per liter of plasma in the mammal. When 150 μg is administered intranasally, the concentration of the Y2 receptor-binding peptide in blood plasma of the mammal increases by at least 30 pM. When 200 μg is administered intranasally, the concentration of the Y2 receptor-binding peptide in blood plasma of the mammal increases by at least 60 pM. In preferred embodiments, the elevated concentrations of the Y2-receptor-binding peptide remains elevated in the plasma of the mammal for at least 30 minutes, preferably at least 60 minutes following a single intranasal dose of the Y2 receptor-binding peptide. Preferably the mammal is a human.

An effective dose of peptide YY for mucosal administration within the pharmaceutical formulations of the present invention comprises, for example, between about 0.001 pmol to about 100 pmol per kg body weight, between about 0.01 pmol to about 10 pmol per kg body weight, or between about 0.1 pmol to about 5 pmol per kg body weight. In further exemplary embodiments, dosage of peptide YY is between about 0.5 pmol to about 1.0 pmol per kg body weight. In a preferred embodiment an intranasal dose will range from 40 μg to 200 μg, or from 45 μg to 150 μg, from about 50 μg to 100 μg, from about 55 μg to about 85 μg, from 65 μg to about 75 μg, from 45 μg to about 55 μg. The pharmaceutical formulations of the present invention may be administered one or more times per day (for example, before a meal), or 3 times per week or once per week for between one week and at least 96 weeks or even for the life of the individual patient or subject. In certain embodiments, the pharmaceutical formulations of the invention are administered one or more times daily, two times daily, four times daily, six times daily, or eight times daily. Preferably, the Y2 receptor-binding peptide should be administered from 30 to 90 minutes prior to eating.

Intranasal delivery-enhancing agents are employed which enhance delivery of peptide YY into or across a nasal mucosal surface. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The intranasal delivery-enhancing agent of the present invention may be a pH control agent. The pH of the pharmaceutical formulation of the present invention is a factor affecting absorption of peptide YY via paracellular and transcellular pathways to drug transport. In one embodiment, the pharmaceutical formulation of the present invention is pH adjusted to between about pH 3.0 to 6.5. In a further embodiment, the pharmaceutical formulation of the present invention is pH adjusted to between about pH 3.0 to 5.0. In a further embodiment, the pharmaceutical formulation of the present invention is pH adjusted to between about pH 4.0 to 5.0. Generally, the pH is 4.5±0.5.

The instant invention also describes the surprising ability to successfully aerosolize the Y2 receptor binding compound, PYY(3-36), from an aqueous formulation

As noted above, the present invention provides improved methods and compositions for mucosal delivery of Y2 receptor-binding peptide to mammalian subjects for treatment or prevention of a variety of diseases and conditions. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

### Y2 Receptor-Binding Peptides

The Y2 receptor-binding peptides used in the mucosal formulations of the present invention include the pancreatic polypeptide family.” as used herein, is comprised of three naturally occurring bioactive peptide families, PP, NPY, and PYY. Examples of Y2 receptor-binding peptides and their uses are described in U.S. Pat. No. 5,026,685; U.S. Pat. No. 5,574,010; U.S. Pat. No. 5,604,203; U.S. Pat. No. 5,696,093; U.S. Pat. No. 6,046,167; Gehlert et. al., Proc Soc Exp Biol Med 218:7-22 (1998); Sheikh et al. Am J Physiol, 261:701-15(1991); Fournier et al., Mol Pharmacol 45:93-101 (1994); Kirby et al., J Med Chem 38:4579-4586 (1995); Rist et al., Eur J Biochem 247:1019-1028 (1997); Kirby et al., J Med Chem 36:3802-3808 (1993); Grundemar et al., Regulatory Peptides 62: 131-136 (1996); U.S. Pat. No. 5,696,093 (examples of PYY agonists), U.S. Pat. No. 6,046,167. According to the present invention a Y2 receptor-binding peptide includes the free bases, acid addition salts or metal salts, such as potassium or sodium salts or the peptides Y2 receptor-binding peptides that have been modified by such processes as amidation, glycosylation, acylation, sulfation, phosphorylation, acetylation and cyclization, (U.S. Pat. No. 6,093,692; and U.S. Pat. No. 6,225,445 and pegylation.

### Peptide YY Agonists

As used herein, “PYY” refers to PYY(1-36) in native-sequence or in variant form, as well as derivatives, fragments, and analogs of PYY from any source, whether natural, synthetic, or recombinant. The PYY must be comprised at least the last 15 amino acid residues or analogues thereof of the PYY sequence, PYY(22-36) (SEQ ID NO: 3). Other PYY peptides, which may be used are PYY(1-36) (SEQ ID NO: 1) PYY(3-36) SEQ ID NO: 2) PYY(4-36) (SEQ ID NO:4) PYY(5-36) (SEQ ID NO: 5), PYY(6-36) (SEQ ID NO:6), PYY(7-36) (SEQ ID NO:7) PYY(8-36) (SEQ ID NO: 8), PYY9-36 (SEQ ID NO: 9) PYY(10-36) (SEQ ID NO: 10), PYY(11-36) (SEQ ID NO: 11), PYY(12-36) (SEQ ID NO: 12), PYY(13-36) (SEQ ID NO:13), PYY(14-36) (SEQ ID NO: 14), PYY(15-36) (SEQ ID NO: 15), PYY(16-36) (SEQ ID NO: 16), PYY(17-36) (SEQ ID NO: 17), PYY(18-36) (SEQ ID NO: 18), PYY(19-36) (SEQ ID NO: 19), PYY(20-36) (SEQ ID NO: 20) and PYY(21-36) (SEQ ID NO: 21). These peptides typically bind to the Y receptors in the brain and elsewhere, especially the Y2 and/or Y5 receptors. Typically these peptides are synthesized in endotoxin-free or pyrogen-free forms although this is not always necessary.

Other PYY peptides include those PYY peptides in which conservative amino acid residue changes have been made, for example, site specific mutation of a PYY peptide including [Asp¹⁵] PYY(15-36) (SEQ ID NO: 90), [Thr¹³] PYY(13-36) (SEQ ID NO: 91), [Val¹²] PYY(12-36) (SEQ ID NO: 92), [Glu¹¹] PYY(11-36) (SEQ ID NO: 93), [Asp¹⁰] PYY(10-36) (SEQ ID NO: 94), [Val⁷] PYY(7-36) (SEQ ID NO: 95), [Asp⁶] PYY(6-36) (SEQ ID NO: 96), [Gln⁴] PYY(4-36) (SEQ ID NO: 97), [Arg⁴] PYY(4-36) (SEQ ID NO: 98), [Asn⁴] PYY(4-36) (SEQ ID NO: 99), [Val³] PYY(3-36) (SEQ ID NO: 100) and [Leu³] PYY(3-36) (SEQ ID NO: 101). Other PYY peptides include those peptides in which at least two conservative amino acid residue changes have been made including [Asp¹⁰, Asp¹⁵] PYY(10-36) (SEQ ID NO: 102), [Asp⁶, Thr¹³] PYY(6-36) (SEQ ID NO: 103), [Asn⁴, Asp¹⁵] PYY(4-36) (SEQ ID NO: 104) and [Leu³, Asp¹⁰] PYY(3-36) (SEQ ID NO: 105. Also included are analogues of a PYY for example those disclosed in U.S. Pat. Nos. 5,604,203 and 5,574,010; Balasubramaniam, et al., Peptide Research 1: 32 (1988); Japanese Patent Application 2,225,497 (1990); Balasubramaniam, et al., Peptides 14: 1011, 1993; Grandt, et at., Reg. Peptides 51: 151, (1994); PCT International Application 94/03380, U.S. Pat. Nos. 5,604,203 and 5,574,010. These peptides typically bind to the Y receptors in the brain and elsewhere, especially the Y2 and/or Y5 receptors. Typically these peptides are synthesized in endotoxin-free or pyrogen-free forms although this is not always necessary.

PYY agonists include rat PYY (SEQ ID NO: 72) and the amino terminus truncated forms corresponding to the human, pig PYY (SEQ ID NO: 73) and the amino terminus truncated forms corresponding to the human and guinea pig PYY (SEQ ID NO: 74) and the amino terminus truncated forms corresponding to the human.

According to the present invention a PYY peptide also includes the free bases, acid addition salts or metal salts, such as potassium or sodium salts of the peptides, and PYY peptides that have been modified by such processes as amidation, glycosylation, acylation, sulfation, phosphorylation, acetylation, cyclization and other well known covalent modification methods. These peptides typically bind to the Y receptors in the brain and elsewhere, especially the Y2 and/or Y5 receptors. Typically these peptides are synthesized in endotoxin-free or pyrogen-free forms although this is not always necessary.

### Neuropeptide Y Agonists

NPY is another Y2 receptor-binding peptide. NPY peptides include full-length NPY(1-36) (SEQ ID NO: 22) as well as well as fragments of NPY(1-36), which have been truncated at the amino terminus. To be effective in binding the Y2 receptor, the NPY agonist should have at least the last 11 amino acid residues at the carboxyl terminus, i.e., be comprised of NPY(26-36) (SEQ ID NO: 23). Other examples of NPY agonists that bind to the Y2 receptor are NPY(3-36) (SEQ ID NO: 24), NPY(4-36) (SEQ ID NO: 25), NPY(5-36) (SEQ ID NO: 26), NPY(6-36) (SEQ ID NO: 27), NPY(7-36) (SEQ ID NO: 28), NPY(8-36) (SEQ ID NO: 29), NPY(9-36) (SEQ ID NO: 30), NPY(10-36) (SEQ ID NO: 31), NPY(1′-36) (SEQ ID NO: 32), NPY(12-36) (SEQ ID NO: 33), NPY(13-36) (SEQ ID NO: 34), NPY(14-36) (SEQ ID NO: 35), NPY(15-36) (SEQ ID NO: 36), NPY(16-36) (SEQ ID NO: 37), NPY(17-36) (SEQ ID NO: 38), NPY(18-36) (SEQ ID NO: 39), NPY(19-36) (SEQ ID NO: 40), NPY(20-36) (SEQ ID NO: 41), NPY(21-36) (SEQ ID NO: 42), NPY(22-36) (SEQ ID NO: 43), NPY(23-36) (SEQ ID NO: 44), NPY(24-36) (SEQ ID NO: 45) and NPY(25-36) (SEQ ID NO: 46).

Other NPY agonists include rat NPY (SEQ ID NO: 75) and the amino terminus truncated forms from NPY(3-36) to NPY(26-36) as in the human form, rabbit NPY (SEQ ID NO: 76) and the amino terminus truncated forms from NPY(3-36) to NPY(26-36) as in the human form, dog NPY (SEQ ID NO: 77) and the amino terminus truncated forms NPY(3-36) to NPY(26-36) as in the human form, pig NPY (SEQ ID NO: 78) and the amino terminus truncated forms from NPY(3-36) to NPY(26-36) as in the human form, cow NPY (SEQ ID NO: 79) and the amino terminus truncated forms from NPY(3-36) to NPY26-36 as in the human form, sheep NPY (SEQ ID NO:80) and the amino terminus truncated forms from NPY(3-36) to NPY(26-36) as in the human form and guinea pig (SEQ 81) and the amino terminus truncated forms from NPY(3-36) to NPY(26-36) as in the human form.

According to the present invention a NPY peptide also includes the free bases, acid addition salts or metal salts, such as potassium or sodium salts of the peptides, and NPY peptides that have been modified by such processes as amidation, glycosylation, acylation, sulfation, phosphorylation, acetylation, cyclization and other known covalent modification methods. These peptides typically bind to the Y receptors in the brain and elsewhere, especially the Y2 and/or Y5 receptors. Typically these peptides are synthesized in endotoxin-free or pyrogen-free forms although this is not always necessary.

### Pancreatic Peptide

Pancreatic Peptide (PP) and PP agonist also bind to the Y2 receptor. Examples of the PP agonists are the full-length PP(1-36) (SEQ ID NO: 47) and a number of PP fragments, which are truncated at the amino-terminus. To bind to the Y2 receptor the PP agonist must have the last 11 amino acid residues at the carboxyl-terminus, PP(26-36), (SEQ ID NO: 48). Examples of other PP, which bind to the Y2 receptor, are PP(3-36) (SEQ ID NO: 49), PP(4-36) (SEQ ID NO: 50), PP(5-36) (SEQ ID NO: 51), PP(6-36) (SEQ ID NO: 52), PP(7-36) (SEQ ID NO: 53), PP(8-36) (SEQ ID NO: 54), PP(9-36) (SEQ ID NO: 55), PP(10-36) (SEQ ID NO: 56), PP(11-36) (SEQ ID NO: 57), PP(12-36) (SEQ ID NO: 58), PP(13-36) (SEQ ID NO: 59), PP(14-36) (SEQ ID NO: 60), PP(15-36) (SEQ ID NO: 61), PP(16-36) (SEQ ID NO: 62), PP(17-36) (SEQ ID NO: 63), PP(18-36) (SEQ ID NO: 64), PP(19-36) (SEQ ID NO: 65), PP(20-36) (SEQ ID NO: 66), PP(21-36) (SEQ ID NO: 67), PP(22-36) (SEQ ID NO: 68), PP(23-36) (SEQ ID NO: 69), PP(24-36) (SEQ ID NO: 70) and PP(25-36) (SEQ ID NO: 71).

Other PP agonists include sheep PP (SEQ ID NO: 82) and the amino terminus truncated forms from PP(3-36) to PP(26-36) as in the human form, pig PP (SEQ ID NO: 83) and the amino terminus truncated forms from PP(3-36) to PP(26-36) as in the human form, dog PP (SEQ ID NO: 84) and the amino terminus truncated forms PP(3-36) to PP(26-36) as in the human form, cat PP (SEQ ID NO: 85) and the amino terminus truncated forms from PP(3-36) to PP(26-36) as in the human form, cow PP (SEQ ID NO: 86) and the amino terminus truncated forms from PP(3-36) to PP(26-36) as in the human form, rat PP (SEQ ID NO:87) and the amino terminus truncated forms from PP(3-36) to PP(26-36) as in the human form, mouse (SEQ 88) and the amino terminus truncated forms from PP(3-36) to PP(26-36) as in the human form, and guinea pig PP (SEQ ID NO: 89).

According to the present invention a PP peptide also includes the free bases, acid addition salts or metal salts, such as potassium or sodium salts of the peptides, and PP peptides that have been modified by such processes as amidation, glycosylation, acylation, sulfation, phosphorylation, acetylation, cyclization, and other known covalent modification methods. These peptides typically bind to the Y receptors in the brain and elsewhere, especially the Y2 and/or Y5 receptors. Typically these peptides are synthesized in endotoxin-free or pyrogen-free forms although this is not always necessary.

### Mucosal Delivery Enhancing Agents

“Mucosal delivery enhancing agents” are defined as chemicals and other excipients that, when added to a formulation comprising water, salts and/or common buffers and Y2 receptor-binding peptide (the control formulation) produce a formulation that produces a significant increase in transport of Y2 receptor-binding peptide across a mucosa as measured by the maximum blood, serum, or cerebral spinal fluid concentration (Cₘₐₓ) or by the area under the curve, AUC, in a plot of concentration versus time. A mucosa includes the nasal, oral, intestinal, buccal, bronchopulmonary, vaginal, and rectal mucosal surfaces and in fact includes all mucus-secreting membranes lining all body cavities or passages that communicate with the exterior. Mucosal delivery enhancing agents are sometimes called carriers.

### Endotoxin-Free Formulation

“Endotoxin-free formulation” means a formulation which contains a Y2-receptor-binding peptide and one or more mucosal delivery enhancing agents that is substantially free of endotoxins and/or related pyrogenic substances. Endotoxins include toxins that are confined inside a microorganism and are released only when the microorganisms are broken down or die. Pyrogenic substances include fever-inducing, thermostable substances (glycoproteins) from the outer membrane of bacteria and other microorganisms. Both of these substances can cause fever, hypotension and shock if administered to humans. Producing formulations that are endotoxin-free can require special equipment, expert artisans, and can be significantly more expensive than making formulations that are not endotoxin-free. Because intravenous administration of NPY or PYY simultaneously with infusion of endotoxin in rodents has been shown to prevent the hypotension and even death associated with the administration of endotoxin alone (U.S. Pat. No. 4,839,343), producing endotoxin-free formulations of these therapeutic agents would not be expected to be necessary for non-parental (non-injected) administration.

### Non-Infused Administration

“Non-infused administration” means any method of delivery that does not involve an injection directly into an artery or vein, a method which forces or drives (typically a fluid) into something and especially to introduce into a body part by means of a needle, syringe or other invasive method. Non-infused administration includes subcutaneous injection, intramuscular injection, intraperitoneal injection and the non-injection methods of delivery to a mucosa.

### Treatment and Prevention of Obesity

As noted above, the instant invention provides improved and useful methods and compositions for nasal mucosal delivery of a Y2 receptor-binding peptide to prevent and treat obesity in mammalian subjects. As used herein, prevention and treatment of obesity mean prevention of the onset or lowering the incidence or severity of clinical obesity by reducing food intake during meals and/or reducing body weight during administration or maintaining a reduced body weight following weight loss or before weight gain has occurred.

The instant invention provides improved and useful methods and compositions for nasal mucosal delivery of Y2 receptor-binding peptide to regions of the brain, for example, the hypothalamus or the proopiomelanocortin (POMC) and NPY arcuate neurons, to prevent and treat obesity in mammalian subjects. The Y2 receptor-binding peptide can also be administered in conjunction with a Y1 receptor antagonist such as dihyropyridine.

Methods and Compositions of Delivery

Improved methods and compositions for mucosal administration of Y2 receptor-binding peptide to mammalian subjects optimize Y2 receptor-binding peptide dosing schedules. The present invention provides mucosal delivery of Y2 receptor-binding peptide formulated with one or more mucosal delivery-enhancing agents wherein Y2 receptor-binding peptide dosage release is substantially normalized and/or sustained for an effective delivery period of Y2 receptor-binding peptide release ranges from approximately 0.1 to 2.0 hours; 0.4 to 1.5 hours; 0.7 to 1.5 hours; or 0.8 to 1.0 hours; following mucosal administration. The sustained release of Y2 receptor-binding peptide achieved may be facilitated by repeated administration of exogenous Y2 receptor-binding peptide utilizing methods and compositions of the present invention.

Compositions and Methods of Sustained Release

Improved compositions and methods for mucosal administration of Y2 receptor-binding peptide to mammalian subjects optimize Y2 receptor-binding peptide dosing schedules. The present invention provides improved mucosal (e.g., nasal) delivery of a formulation comprising Y2 receptor-binding peptide in combination with one or more mucosal delivery-enhancing agents and an optional sustained release-enhancing agent or agents. Mucosal delivery-enhancing agents of the present invention yield an effective increase in delivery, e.g., an increase in the maximal plasma concentration (Cₘₐₓ) to enhance the therapeutic activity of mucosally-administered Y2 receptor-binding peptide. A second factor affecting therapeutic activity of Y2 receptor-binding peptide in the blood plasma and CNS is residence time (RT). Sustained release-enhancing agents, in combination with intranasal delivery-enhancing agents, increase Cₘₐₓ and increase residence time (RT) of Y2 receptor-binding peptide. Polymeric delivery vehicles and other agents and methods of the present invention that yield sustained release-enhancing formulations, for example, polyethylene glycol (PEG), are disclosed herein. The present invention provides an improved Y2 receptor-binding peptide delivery method and dosage form for treatment of symptoms related to obesity, colon cancer, pancreatic cancer, or breast cancer in mammalian subjects.

Within the mucosal delivery formulations and methods of the invention, the Y2 receptor-binding peptide is frequently combined or coordinately administered with a suitable carrier or vehicle for mucosal delivery. As used herein, the term “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, can be found in the U.S. Pharmacopeia National Formulary, 1857-1859, (1990). Some examples of the materials which can serve as pharmaceutically acceptable carriers are gelatin; talc; excipients such as cocoa butter and suppository waxes; oils such as peanut oil, cottonseed oil, safflower oil, sesame oil, olive oil, corn oil and soybean oil; glycols, such as propylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

Within the mucosal delivery compositions and methods of the invention, various delivery-enhancing agents are employed which enhance delivery of Y2 receptor-binding peptide into or across a mucosal surface. In this regard, delivery of Y2 receptor-binding peptide across the mucosal epithelium can occur “transcellularly” or “paracellularly”. The extent to which these pathways contribute to the overall flux and bioavailability of the Y2 receptor-binding peptide depends upon the environment of the mucosa, the physico-chemical properties the active agent, and on the properties of the mucosal epithelium. Paracellular transport involves only passive diffusion, whereas transcellular transport can occur by passive, facilitated or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected Y2 receptor-binding peptide within the invention. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against macromolecular permeation would suggest that the paracellular route would be a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively or within a single method or composition.

As used herein, “mucosal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of Y2 receptor-binding peptide or other biologically active compound(s). Enhancement of mucosal delivery can thus occur by any of a variety of mechanisms, for example by increasing the diffusion, transport, persistence or stability of Y2 receptor-binding peptide, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, a “mucosally effective amount of Y2 receptor-binding peptide” contemplates effective mucosal delivery of Y2 receptor-binding peptide to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of Y2 receptor-binding peptide in a blood plasma”, “area under concentration vs. time curve (AUC) of Y2 receptor-binding peptide in a blood plasma”, “time to maximal plasma concentration (tₘₐₓ) of Y2 receptor-binding peptide in a blood plasma” are pharmacokinetic parameters known to one skilled in the art. Laursen et al., Eur. J. Endocrinology, 135: 309-315, 1996. The “concentration vs. time curve” measures the concentration of Y2 receptor-binding peptide in a blood serum of a subject vs. time after administration of a dosage of Y2 receptor-binding peptide to the subject either by intranasal, intramuscular, subcutaneous, or other parenteral route of administration. “Cₘₐₓ” is the maximum concentration of Y2 receptor-binding peptide in the blood serum of a subject following a single dosage of Y2 receptor-binding peptide to the subject. “tₘₐₓ” is the time to reach maximum concentration of Y2 receptor-binding peptide in a blood serum of a subject following administration of a single dosage of Y2 receptor-binding peptide to the subject.

As used herein, “area under concentration vs. time curve (AUC) of Y2 receptor-binding peptide in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of intramuscular, subcutaneous, intravenous and intranasal Y2 receptor-binding peptide administrations were performed by analysis of variance (ANOVA). For pair wise comparisons a Bonferroni-Holmes sequential procedure was used to evaluate significance. The dose-response relationship between the three nasal doses was estimated by regression analysis. P<0.05 was considered significant. Results are given as mean values +/−SEM.

While the mechanism of absorption promotion may vary with different mucosal delivery-enhancing agents of the invention, useful reagents in this context will not substantially adversely affect the mucosal tissue and will be selected according to the physicochemical characteristics of the particular Y2 receptor-binding peptide or other active or delivery-enhancing agent. In this context, delivery-enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption-promoting agents for coordinate administration or combinatorial formulation with Y2 receptor-binding peptide of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium lauryl sulfate, oleic acid, and the bile salts, may be employed to enhance mucosal penetration of the Y2 receptor-binding peptide. In additional aspects, surfactants (e.g., polysorbates) are employed as adjunct compounds, processing agents, or formulation additives to enhance intranasal delivery of the Y2 receptor-binding peptide. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the Y2 receptor-binding peptide from the vehicle into the mucosa.

Additional mucosal delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the Y2 receptor-binding peptide. These include, inter alia, α, β, or γ-cyclodextrins and β-cyclodextrin derivatives (e.g., 2-hydroxypropyl-β-cyclodextrin and heptakis(2,6-di-O-methyl-β-cyclodextrin) methylated cyclodextrins (methyl-β-cyclodextrin and dimethyl-β-cyclodextrin), ethylated cyclodextrins, hydroxypropylated cyclodextrins, and polymeric cyclodextrins. These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylacetic compositions of the present invention may be supplemented with any suitable penetration-promoting agent that facilitates absorption, diffusion, or penetration of Y2 receptor-binding peptide across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxylic acids such as glycine, alanine, phenylalanine, proline, hydroxyproline, etc.; hydroxyamino acids such as serine; acidic amino acids such as aspartic acid, glutamic acid, etc; and basic amino acids such as lysine etc-inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lacetic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved nasal mucosal delivery formulations and methods are provided that allow delivery of Y2 receptor-binding peptide and other therapeutic agents within the invention across mucosal barriers between administration and selected target sites. Certain formulations are specifically adapted for a selected target cell, tissue or organ, or even a particular disease state. In other aspects, formulations and methods provide for efficient, selective endo- or transcytosis of Y2 receptor-binding peptide specifically routed along a defined intracellular or intercellular pathway. Typically, the Y2 receptor-binding peptide is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The Y2 receptor-binding peptide may be provided in a delivery vehicle or otherwise modified (e.g., in the form of a prodrug), wherein release or activation of the Y2 receptor-binding peptide is triggered by a physiological stimulus (e.g. pH change, lysosomal enzymes, etc.) Often, the Y2 receptor-binding peptide is pharmacologically inactive until it reaches its target site for activity. In most cases, the Y2 receptor-binding peptide and other formulation components are non-toxic and non-immunogenic. In this context, carriers and other formulation components are generally selected for their ability to be rapidly degraded and excreted under physiological conditions. At the same time, formulations are chemically and physically stable in dosage form for effective storage.

Peptide and Protein Analogs and Mimetics

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. A wide variety of useful analogs and mimetics of Y2 receptor-binding peptide are contemplated for use within the invention and can be produced and tested for biological activity according to known methods. Often, the peptides or proteins of Y2 receptor-binding peptide or other biologically active peptides or proteins for use within the invention are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine. By aligning a peptide or protein analog optimally with a corresponding native peptide or protein, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide or protein.

An approach for stabilizing solid protein formulations of the invention is to increase the physical stability of purified, e.g., lyophilized, protein. This will inhibit aggregation via hydrophobic interactions as well as via covalent pathways that may increase as proteins unfold. Stabilizing formulations in this context often include polymer-based formulations, for example a biodegradable hydrogel formulation/delivery system. As noted above, the critical role of water in protein structure, function, and stability is well known. Typically, proteins are relatively stable in the solid state with bulk water removed. However, solid therapeutic protein formulations may become hydrated upon storage at elevated humidity or during delivery from a sustained release composition or device. The stability of proteins generally drops with increasing hydration. Water can also play a significant role in solid protein aggregation, for example, by increasing protein flexibility resulting in enhanced accessibility of reactive groups, by providing a mobile phase for reactants, and by serving as a reactant in several deleterious processes such as beta-elimination and hydrolysis.

Protein preparations containing between about 6% to 28% water are the most unstable. Below this level, the mobility of bound water and protein internal motions are low. Above this level, water mobility and protein motions approach those of full hydration. Up to a point, increased susceptibility toward solid-phase aggregation with increasing hydration has been observed in several systems. However, at higher water content, less aggregation is observed because of the dilution effect.

In accordance with these principles, an effective method for stabilizing peptides and proteins against solid-state aggregation for mucosal delivery is to control the water content in a solid formulation and maintain the water activity in the formulation at optimal levels. This level depends on the nature of the protein, but in general, proteins maintained below their “monolayer” water coverage will exhibit superior solid-state stability.

A variety of additives, diluents, bases and delivery vehicles are provided within the invention that effectively controls water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto

Certain additives also impart significant physical stability to dry, e.g., lyophilized proteins. These additives can also be used within the invention to protect the proteins against aggregation not only during lyophilization but also during storage in the dry state.

Various additional preparative components and methods, as well as specific formulation additives, are provided herein which yield formulations for mucosal delivery of aggregation-prone peptides and proteins, wherein the peptide or protein is stabilized in a substantially pure, unaggregated form using a solubilization agent. A range of components and additives are contemplated for use within these methods and formulations. Exemplary of these solubilization agents are cyclodextrins (CDs), which selectively bind hydrophobic side chains of polypeptides. These CDs have been found to bind to hydrophobic patches of proteins in a manner that significantly inhibits aggregation. This inhibition is selective with respect to both the CD and the protein involved. Such selective inhibition of protein aggregation provides additional advantages within the intranasal delivery methods and compositions of the invention. Additional agents for use in this context include CD dimers, trimers and tetramers with varying geometries controlled by the linkers that specifically block aggregation of peptides and protein. Yet solubilization agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention.

Charge Modifying and pH Control Agents and Methods

To improve the transport characteristics of biologically active agents (including Y2 receptor-binding peptide, other active peptides and proteins, and macromolecular and small molecule drugs) for enhanced delivery across hydrophobic mucosal membrane barriers, the invention also provides techniques and reagents for charge modification of selected biologically active agents or delivery-enhancing agents described herein. In this regard, the relative permeability of macromolecules is generally related to their partition coefficients. The degree of ionization of molecules, which is dependent on the pKₐ of the molecule and the pH at the mucosal membrane surface, also affects permeability of the molecules. Permeation and partitioning of biologically active agents, including Y2 receptor-binding peptide and analogs of the invention, for mucosal delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

Consistent with these general teachings, mucosal delivery of charged macromolecular species, including Y2 receptor-binding peptide and other biologically active peptides and proteins, within the methods and compositions of the invention is substantially improved when the active agent is delivered to the mucosal surface in a substantially un-ionized, or neutral, electrical charge state.

Certain Y2 receptor-binding peptide and other biologically active peptide and protein components of mucosal formulations for use within the invention will be charge modified to yield an increase in the positive charge density of the peptide or protein. These modifications extend also to cationization of peptide and protein conjugates, carriers and other delivery forms disclosed herein. Cationization offers a convenient means of altering the biodistribution and transport properties of proteins and macromolecules within the invention. Cationization is undertaken in a manner that substantially preserves the biological activity of the active agent and limits potentially adverse side effects, including tissue damage and toxicity.

Degradative Enzyme Inhibitory Agents and Methods

Another excipient that may be included in a trans-mucosal preparation is a degradative enzyme inhibitor. Exemplary mucoadhesive polymer-enzyme inhibitor complexes that are useful within the mucosal delivery formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase). As described in further detail below, certain embodiments of the invention will optionally incorporate a novel chitosan derivative or chemically modified form of chitosan. One such novel derivative for use within the invention is denoted as a β-[1→4]-2-guanidino-2-deoxy-D-glucose polymer (poly-GuD).

Any inhibitor that inhibits the activity of an enzyme to protect the biologically active agent(s) may be usefully employed in the compositions and methods of the invention. Useful enzyme inhibitors for the protection of biologically active proteins and peptides include, for example, soybean trypsin inhibitor, pancreatic trypsin inhibitor, chymotrypsin inhibitor and trypsin and chrymotrypsin inhibitor isolated from potato (solanum tuberosum L.) tubers. A combination or mixtures of inhibitors may be employed. Additional inhibitors of proteolytic enzymes for use within the invention include ovomucoid-enzyme, gabaxate mesylate, alpha1-antitrypsin, aprotinin, amastatin, bestatin, puromycin, bacitracin, leupepsin, alpha2-macroglobulin, pepstatin and egg white or soybean trypsin inhibitor. These and other inhibitors can be used alone or in combination. The inhibitor(s) may be incorporated in or bound to a carrier, e.g., a hydrophilic polymer, coated on the surface of the dosage form which is to contact the nasal mucosa, or incorporated in the superficial phase of the surface, in combination with the biologically active agent or in a separately administered (e.g., pre-administered) formulation.

The amount of the inhibitor, e.g., of a proteolytic enzyme inhibitor that is optionally incorporated in the compositions of the invention will vary depending on (a) the properties of the specific inhibitor, (b) the number of functional groups present in the molecule (which may be reacted to introduce ethylenic unsaturation necessary for copolymerization with hydrogel forming monomers), and (c) the number of lectin groups, such as glycosides, which are present in the inhibitor molecule. It may also depend on the specific therapeutic agent that is intended to be administered. Generally speaking, a useful amount of an enzyme inhibitor is from about 0.1 mg/ml to about 50 mg/ml, often from about 0.2 mg/ml to about 25 mg/ml, and more commonly from about 0.5 mg/ml to 5 mg/ml of the of the formulation (i.e., a separate protease inhibitor formulation or combined formulation with the inhibitor and biologically active agent).

In the case of trypsin inhibition, suitable inhibitors may be selected from, e.g., aprotinin, BBI, soybean trypsin inhibitor, chicken ovomucoid, chicken ovoinhibitor, human pancreatic trypsin inhibitor, camostat mesilate, flavonoid inhibitors, antipain, leupeptin, p-aminobenzamidine, AEBSF, TLCK (tosyllysine chloromethylketone), APMSF, DFP, PMSF, and poly(acrylate) derivatives. In the case of chymotrypsin inhibition, suitable inhibitors may be selected from, e.g., aprotinin, BBI, soybean trypsin inhibitor, chymostatin, benzyloxycarbonyl-Pro-Phe-CHO, FK-448, chicken ovoinhibitor, sugar biphenylboronic acids complexes, DFP, PMSF, β-phenylpropionate, and poly(acrylate) derivatives. In the case of elastase inhibition, suitable inhibitors may be selected from, e.g., elastatinal, methoxysuccinyl-Ala-Ala-Pro-Val-chloromethylketone (MPCMK), BBI, soybean trypsin inhibitor, chicken ovoinhibitor, DFP, and PMSF.

Additional enzyme inhibitors for use within the invention are selected from a wide range of non-protein inhibitors that vary in their degree of potency and toxicity. As described in further detail below, immobilization of these adjunct agents to matrices or other delivery vehicles, or development of chemically modified analogues, may be readily implemented to reduce or even eliminate toxic effects, when they are encountered. Among this broad group of candidate enzyme inhibitors for use within the invention are organophosphorous inhibitors, such as diisopropylfluorophosphate (DFP) and phenylmethylsulfonyl fluoride (PMSF), which are potent, irreversible inhibitors of serine proteases (e.g., trypsin and chymotrypsin). The additional inhibition of acetylcholinesterase by these compounds makes them highly toxic in uncontrolled delivery settings. Another candidate inhibitor, 4-(2-Aminoethyl)-benzenesulfonyl fluoride (AEBSF), has an inhibitory activity comparable to DFP and PMSF, but it is markedly less toxic. (4-Aminophenyl)-methanesulfonyl fluoride hydrochloride (APMSF) is another potent inhibitor of trypsin, but is toxic in uncontrolled settings. In contrast to these inhibitors, 4-(4-isopropylpiperadinocarbonyl)phenyl 1,2,3,4,-tetrahydro-1-naphthoate methanesulphonate (FK-448) is a low toxic substance, representing a potent and specific inhibitor of chymotrypsin. Further representatives of this non-protein group of inhibitor candidates, and also exhibiting low toxic risk, are camostat mesilate (N,N′-dimethyl carbamoylmethyl-p-(p′-guanidino-benzoyloxy)phenylacetate methane-sulphonate).

Yet another type of enzyme inhibitory agent for use within the methods and compositions of the invention are amino acids and modified amino acids that interfere with enzymatic degradation of specific therapeutic compounds. For use in this context, amino acids and modified amino acids are substantially non-toxic and can be produced at a low cost. However, due to their low molecular size and good solubility, they are readily diluted and absorbed in mucosal environments. Nevertheless, under proper conditions, amino acids can act as reversible, competitive inhibitors of protease enzymes. Certain modified amino acids can display a much stronger inhibitory activity. A desired modified amino acid in this context is known as a ‘transition-state’ inhibitor. The strong inhibitory activity of these compounds is based on their structural similarity to a substrate in its transition-state geometry, while they are generally selected to have a much higher affinity for the active site of an enzyme than the substrate itself. Transition-state inhibitors are reversible, competitive inhibitors. Examples of this type of inhibitor are α-aminoboronic acid derivatives, such as boro-leucine, boro-valine and boro-alanine. The boron atom in these derivatives can form a tetrahedral boronate ion that is believed to resemble the transition state of peptides during their hydrolysis by aminopeptidases. These amino acid derivatives are potent and reversible inhibitors of aminopeptidases and it is reported that boro-leucine is more than 100-times more effective in enzyme inhibition than bestatin and more than 1000-times more effective than puromycin. Another modified amino acid for which a strong protease inhibitory activity has been reported is N-acetylcysteine, which inhibits enzymatic activity of aminopeptidase N. This adjunct agent also displays mucolytic properties that can be employed within the methods and compositions of the invention to reduce the effects of the mucus diffusion barrier.

Still other useful enzyme inhibitors for use within the coordinate administration methods and combinatorial formulations of the invention may be selected from peptides and modified peptide enzyme inhibitors. An important representative of this class of inhibitors is the cyclic dodecapeptide, bacitracin, obtained from Bacillus licheniformis. In addition to these types of peptides, certain dipeptides and tripeptides display weak, non-specific inhibitory activity towards some protease. By analogy with amino acids, their inhibitory activity can be improved by chemical modifications. For example, phosphinic acid dipeptide analogues are also ‘transition-state’ inhibitors with a strong inhibitory activity towards aminopeptidases. They have reportedly been used to stabilize nasally administered leucine enkephalin. Another example of a transition-state analogue is the modified pentapeptide pepstatin, which is a very potent inhibitor of pepsin. Structural analysis of pepstatin, by testing the inhibitory activity of several synthetic analogues, demonstrated the major structure-function characteristics of the molecule responsible for the inhibitory activity. Another special type of modified peptide includes inhibitors with a terminally located aldehyde function in their structure. For example, the sequence benzyloxycarbonyl-Pro-Phe-CHO, which fulfills the known primary and secondary specificity requirements of chymotrypsin, has been found to be a potent reversible inhibitor of this target proteinase. The chemical structures of further inhibitors with a terminally located aldehyde function, e.g. antipain, leupeptin, chymostatin and elastatinal, are also known in the art, as are the structures of other known, reversible, modified peptide inhibitors, such as phosphoramidon, bestatin, puromycin and amastatin.

Due to their comparably high molecular mass, polypeptide protease inhibitors are more amenable than smaller compounds to concentrated delivery in a drug-carrier matrix. Additional agents for protease inhibition within the formulations and methods of the invention involve the use of complexing agents. These agents mediate enzyme inhibition by depriving the intranasal environment (or preparative or therapeutic composition) of divalent cations, which are co-factors for many proteases. For instance, the complexing agents EDTA and DTPA as coordinately administered or combinatorially formulated adjunct agents, in suitable concentration, will be sufficient to inhibit selected proteases to thereby enhance intranasal delivery of biologically active agents according to the invention. Further representatives of this class of inhibitory agents are EGTA, 1,10-phenanthroline and hydroxychinoline. In addition, due to their propensity to chelate divalent cations, these and other complexing agents are useful within the invention as direct, absorption-promoting agents.

As noted in more detail elsewhere herein, it is also contemplated to use various polymers, particularly mucoadhesive polymers, as enzyme inhibiting agents within the coordinate administration, multi-processing and/or combinatorial formulation methods and compositions of the invention. For example, poly(acrylate) derivatives, such as poly(acrylic acid) and polycarbophil, can affect the activity of various proteases, including trypsin, chymotrypsin. The inhibitory effect of these polymers may also be based on the complexation of divalent cations such as Ca²⁺ and Zn⁺. It is further contemplated that these polymers may serve as conjugate partners or carriers for additional enzyme inhibitory agents, as described above. For example, a chitosan-EDTA conjugate has been developed and is useful within the invention that exhibits a strong inhibitory effect towards the enzymatic activity of zinc-dependent proteases. The mucoadhesive properties of polymers following covalent attachment of other enzyme inhibitors in this context are not expected to be substantially compromised, nor is the general utility of such polymers as a delivery vehicle for biologically active agents within the invention expected to be diminished. On the contrary, the reduced distance between the delivery vehicle and mucosal surface afforded by the mucoadhesive mechanism will minimize presystemic metabolism of the active agent, while the covalently bound enzyme inhibitors remain concentrated at the site of drug delivery, minimizing undesired dilution effects of inhibitors as well as toxic and other side effects caused thereby. In this manner, the effective amount of a coordinately administered enzyme inhibitor can be reduced due to the exclusion of dilution effects.

Exemplary mucoadhesive polymer-enzyme inhibitor complexes that are useful within the mucosal formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil—elastatinal (anti-elastase); Chitosan—antipain (anti-trypsin); Poly(acrylic acid)—bacitracin (anti-aminopeptidase N); Chitosan—EDTA (anti-aminopeptidase N, anti-carboxypeptidase A); Chitosan—EDTA—antipain (anti-trypsin, anti-chymotrypsin, anti-elastase).

Mucolytic and Mucus-Clearing Agents and Methods

Effective delivery of biotherapeutic agents via intranasal administration must take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant invention optionally incorporate effective mucolytic or mucus-clearing agents, which serve to degrade, thin or clear mucus from intranasal mucosal surfaces to facilitate absorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited. In contrast, bacterial glycosidases which allow these microorganisms to permeate mucus layers of their hosts.

For combinatorial use with most biologically active agents within the invention, including peptide and protein therapeutics, non-ionogenic detergents are generally also useful as mucolytic or mucus-clearing agents. These agents typically will not modify or substantially impair the activity of therapeutic polypeptides.

Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Ciliostatic agents find use within the methods and compositions of the invention to increase the residence time of mucosally (e.g., intranasally) administered Y2 receptor-binding peptide, analogs and mimetics, and other biologically active agents disclosed herein. In particular, the delivery these agents within the methods and compositions of the invention is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of Y2 receptor-binding peptide, analogs and mimetics, and other biologically active agents disclosed herein, without unacceptable adverse side effects.

Within more detailed aspects, a specific ciliostatic factor is employed in a combined formulation or coordinate administration protocol with one or more Y2 receptor-binding peptide proteins, analogs and mimetics, and/or other biologically active agents disclosed herein. Various bacterial ciliostatic factors isolated and characterized in the literature may be employed within these embodiments of the invention. Ciliostatic factors from the bacterium Pseudomonas aeruginosa include a phenazine derivative, a pyo compound (2-alkyl-4-hydroxyquinolines), and a rhamnolipid (also known as a hemolysin). The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis, which was associated with altered ciliary membranes. More extensive exposure to rhamnolipid was associated with removal of dynein arms from axonemes.

Surface Active Agents and Methods

Within more detailed aspects of the invention, one or more membrane penetration-enhancing agents may be employed within a mucosal delivery method or formulation of the invention to enhance mucosal delivery of Y2 receptor-binding peptide proteins, analogs and mimetics, and other biologically active agents disclosed herein. Membrane penetration enhancing agents in this context can be selected from: (i) a surfactant, (ii) a bile salt, (iii) a phospholipid additive, mixed micelle, liposome, or carrier, (iv) an alcohol, (v) an enamine, (vi) an NO donor compound, (vii) a long-chain amphipathic molecule (viii) a small hydrophobic penetration enhancer; (ix) sodium or a salicylic acid derivative; (x) a glycerol ester of acetoacetic acid (xi) a cyclodextrin or beta-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, or (xviii) an inhibitor of cholesterol synthesis; or (xix) any combination of the membrane penetration enhancing agents recited in (i)-(xix).

Certain surface-active agents are readily incorporated within the mucosal delivery formulations and methods of the invention as mucosal absorption enhancing agents. These agents, which may be coordinately administered or combinatorially formulated with Y2 receptor-binding peptide proteins, analogs and mimetics, and other biologically active agents disclosed herein, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF). The mechanisms of action of these various classes of surface-active agents typically include solubilization of the biologically active agent. For proteins and peptides which often form aggregates, the surface active properties of these absorption promoters can allow interactions with proteins such that smaller units such as surfactant coated monomers may be more readily maintained in solution. Examples of other surface-active agents are L-α-Phosphatidylcholine Didecanoyl (DDPC) polysorbate 80 and polysorbate 20. These monomers are presumably more transportable units than aggregates. A second potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

Degradation Enzymes and Inhibitors of Fatty Acid and Cholesterol Synthesis

In related aspects of the invention, Y2 receptor-binding peptide proteins, analogs and mimetics, and other biologically active agents for mucosal administration are formulated or coordinately administered with a penetration enhancing agent selected from a degradation enzyme, or a metabolic stimulatory agent or inhibitor of synthesis of fatty acids, sterols or other selected epithelial barrier components, U.S. Pat. No. 6,190,894. For example, degradative enzymes such as phospholipase, hyaluronidase, neuraminidase, and chondroitinase may be employed to enhance mucosal penetration of Y2 receptor-binding peptide proteins, analogs and mimetics, and other biologically active agent without causing irreversible damage to the mucosal barrier. In one embodiment, chondroitinase is employed within a method or composition as provided herein to alter glycoprotein or glycolipid constituents of the permeability barrier of the mucosa, thereby enhancing mucosal absorption of Y2 receptor-binding peptide proteins, analogs and mimetics, and other biologically active agents disclosed herein.

With regard to inhibitors of synthesis of mucosal barrier constituents, it is noted that free fatty acids account for 20-25% of epithelial lipids by weight. Two rate-limiting enzymes in the biosynthesis of free fatty acids are acetyl CoA carboxylase and fatty acid synthetase. Through a series of steps, free fatty acids are metabolized into phospholipids. Thus, inhibitors of free fatty acid synthesis and metabolism for use within the methods and compositions of the invention include, but are not limited to, inhibitors of acetyl CoA carboxylase such as 5-tetradecyloxy-2-furancarboxylic acid (TOFA); inhibitors of fatty acid synthetase; inhibitors of phospholipase A such as gomisin A, 2-(p-amylcinnamyl)amino-4-chlorobenzoic acid, bromophenacyl bromide, monoalide, 7,7-dimethyl-5,8-eicosadienoic acid, nicergoline, cepharanthine, nicardipine, quercetin, dibutyryl-cyclic AMP, R-24571, N-oleoylethanolamine, N-(7-nitro-2,1,3-benzoxadiazol-4-yl) phosphostidyl serine, cyclosporine A, topical anesthetics, including dibucaine, prenylamine, retinoids, such as all-trans and 13-cis-retinoic acid, W-7, trifluoperazine, R-24571 (calmidazolium), 1-hexadocyl-3-trifluoroethyl glycero-sn-2-phosphomenthol (MJ33); calcium channel blockers including nicardipine, verapamil, diltiazem, nifedipine, and nimodipine; antimalarials including quinacrine, mepacrine, chloroquine and hydroxychloroquine; beta blockers including propanalol and labetalol; calmodulin antagonists; EGTA; thimersol; glucocorticosteroids including dexamethasone and prednisolone; and nonsteroidal antiinflammatory agents including indomethacin and naproxen.

Free sterols, primarily cholesterol, account for 20-25% of the epithelial lipids by weight. The rate limiting enzyme in the biosynthesis of cholesterol is 3-hydroxy-3-methylglutaryl (HMG) CoA reductase. Inhibitors of cholesterol synthesis for use within the methods and compositions of the invention include, but are not limited to, competitive inhibitors of (HMG) CoA reductase, such as simvastatin, lovastatin, fluindostatin (fluvastatin), pravastatin, mevastatin, as well as other HMG CoA reductase inhibitors, such as cholesterol oleate, cholesterol sulfate and phosphate, and oxygenated sterols, such as 25-OH—and 26-OH—cholesterol; inhibitors of squalene synthetase; inhibitors of squalene epoxidase; inhibitors of DELTA7 or DELTA24 reductases such as 22,25-diazacholesterol, 20,25-diazacholestenol, AY9944, and triparanol.

Each of the inhibitors of fatty acid synthesis or the sterol synthesis inhibitors may be coordinately administered or combinatorially formulated with one or more Y2 receptor-binding peptide proteins, analogs and mimetics, and other biologically active agents disclosed herein to achieve enhanced epithelial penetration of the active agent(s). An effective concentration range for the sterol inhibitor in a therapeutic or adjunct formulation for mucosal delivery is generally from about 0.0001% to about 20% by weight of the total, more typically from about 0.01% to about 5%.

Nitric Oxide Donor Agents and Methods

Within other related aspects of the invention, a nitric oxide (NO) donor is selected as a membrane penetration-enhancing agent to enhance mucosal delivery of one or more Y2 receptor-binding peptide proteins, analogs and mimetics, and other biologically active agents disclosed herein. Various NO donors are known in the art and are useful in effective concentrations within the methods and formulations of the invention. Exemplary NO donors include, but are not limited to, nitroglycerine, nitroprusside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI nd NOR4. Within the methods and compositions of the invention, an effective amount of a selected NO donor is coordinately administered or combinatorially formulated with one or more Y2 receptor-binding peptide proteins, analogs and mimetics, and/or other biologically active agents disclosed herein, into or through the mucosal epithelium.

Agents for Modulating Epithelial Junction Structure and/or Physiology

The present invention provides pharmaceutical composition that contains one or more Y2 receptor-binding peptide proteins, analogs or mimetics, and/or other biologically active agents in combination with mucosal delivery enhancing agents disclosed herein formulated in a pharmaceutical preparation for mucosal delivery.

The permeabilizing agent reversibly enhances mucosal epithelial paracellular transport, typically by modulating epithelial junctional structure and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occludins, or claudins. Examples of this are antibodies, antibody fragments or single-chain antibodies that bind to the extracellular domains of these proteins.

In yet additional detailed embodiments, the invention provides permeabilizing peptides and peptide analogs and mimetics for enhancing mucosal epithelial paracellular transport. The subject peptides and peptide analogs and mimetics typically work within the compositions and methods of the invention by modulating epithelial junctional structure and/or physiology in a mammalian subject. In certain embodiments, the peptides and peptide analogs and mimetics effectively inhibit homotypic and/or heterotypic binding of an epithelial membrane adhesive protein selected from a junctional adhesion molecule (JAM), occludin, or claudin.

One such agent that has been extensively studied is the bacterial toxin from Vibrio cholerae known as the “zonula occludens toxin” (ZOT). This toxin mediates increased intestinal mucosal permeability and causes disease symptoms including diarrhea in infected subjects. Fasano et al., Proc. Nat. Acad. Sci., U.S.A., 8:5242-5246 (1991). When tested on rabbit ileal mucosa, ZOT increased the intestinal permeability by modulating the structure of intercellular tight junctions. More recently, it has been found that ZOT is capable of reversibly opening tight junctions in the intestinal mucosa. It has also been reported that ZOT is capable of reversibly opening tight junctions in the nasal mucosa. U.S. Pat. No. 5,908,825.

Within the methods and compositions of the invention, ZOT, as well as various analogs and mimetics of ZOT that function as agonists or antagonists of ZOT activity, are useful for enhancing intranasal delivery of biologically active agentsby increasing paracellular absorption into and across the nasal mucosa. In this context, ZOT typically acts by causing a structural reorganization of tight junctions marked by altered localization of the junctional protein ZO1. Within these aspects of the invention, ZOT is coordinately administered or combinatorially formulated with the biologically active agent in an effective amount to yield significantly enhanced absorption of the active agent, by reversibly increasing nasal mucosal permeability without substantial adverse side effects

Vasodilator Agents and Methods

Yet another class of absorption-promoting agents that shows beneficial utility within the coordinate administration and combinatorial formulation methods and compositions of the invention are vasoactive compounds, more specifically vasodilators. These compounds function within the invention to modulate the structure and physiology of the submucosal vasculature, increasing the transport rate of Y2 receptor-binding peptide, analogs and mimetics, and other biologically active agents into or through the mucosal epithelium and/or to specific target tissues or compartments (e.g., the systemic circulation or central nervous system.).

Vasodilator agents for use within the invention typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, α-adrenergic and imidazole receptor antagonists, β1-adrenergic agonists, phosphodiesterase inhibitors, eicosanoids and NO donors.

Despite chemical differences, the pharmacokinetic properties of calcium antagonists are similar. Absorption into the systemic circulation is high, and these agents therefore undergo considerable first-pass metabolism by the liver, resulting in individual variation in pharmacokinetics. Except for the newer drugs of the dihydropyridine type (amlodipine, felodipine, isradipine, nilvadipine, nisoldipine and nitrendipine), the half-life of calcium antagonists is short. Therefore, to maintain an effective drug concentration for many of these may require delivery by multiple dosing, or controlled release formulations, as described elsewhere herein. Treatment with the potassium channel opener minoxidil may also be limited in manner and level of administration due to potential adverse side effects.

ACE inhibitors prevent conversion of angiotensin-I to angiotensin-II, and are most effective when renin production is increased. Since ACE is identical to kininase-II, which inactivates the potent endogenous vasodilator bradykinin, ACE inhibition causes a reduction in bradykinin degradation. ACE inhibitors provide the added advantage of cardioprotective and cardioreparative effects, by preventing and reversing cardiac fibrosis and ventricular hypertrophy in animal models. The predominant elimination pathway of most ACE inhibitors is via renal excretion. Therefore, renal impairment is associated with reduced elimination and a dosage reduction of 25 to 50% is recommended in patients with moderate to severe renal impairment.

With regard to NO donors, these compounds are particularly useful within the invention for their additional effects on mucosal permeability. In addition to the above-noted NO donors, complexes of NO with nucleophiles called NO/nucleophiles, or NONOates, spontaneously and nonenzymatically release NO when dissolved in aqueous solution at physiologic pH. In contrast, nitro vasodilators such as nitroglycerin require specific enzyme activity for NO release. NONOates release NO with a defined stoichiometry and at predictable rates ranging from <3 minutes for diethylamine/NO to approximately 20 hours for diethylenetriamine/NO (DETANO).

Within certain methods and compositions of the invention, a selected vasodilator agent is coordinately administered (e.g., systemically or intranasally, simultaneously or in combinatorially effective temporal association) or combinatorially formulated with one or more Y2 receptor-binding peptide, analogs and mimetics, and other biologically active agent(s) in an amount effective to enhance the mucosal absorption of the active agent(s) to reach a target tissue or compartment in the subject (e.g., the liver, hepatic portal vein, CNS tissue or fluid, or blood plasma).

Selective Transport-Enhancing Agents and Methods

The compositions and delivery methods of the invention optionally incorporate a selective transport-enhancing agent that facilitates transport of one or more biologically active agents. These transport-enhancing agents may be employed in a combinatorial formulation or coordinate administration protocol with one or more of the Y2 receptor-binding peptide proteins, analogs and mimetics disclosed herein, to coordinately enhance delivery of one or more additional biologically active agent(s) across mucosal transport barriers, to enhance mucosal delivery of the active agent(s) to reach a target tissue or compartment in the subject (e.g., the mucosal epithelium, liver, CNS tissue or fluid, or blood plasma). Alternatively, the transport-enhancing agents may be employed in a combinatorial formulation or coordinate administration protocol to directly enhance mucosal delivery of one or more of the Y2 receptor-binding peptide proteins, analogs and mimetics, with or without enhanced delivery of an additional biologically active agent.

Exemplary selective transport-enhancing agents for use within this aspect of the invention include, but are not limited to, and binding agents such as lectin binding agents, which are known to interact specifically with epithelial transport barrier components. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the invention. Certain bioadhesive ligands for use within the invention will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more Y2 receptor-binding peptide proteins, analogs and mimetics, and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within the invention. Lectins are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (e.g., concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

In addition to plant lectins, microbial adhesion and invasion factors provide a rich source of candidates for use as adhesive/selective transport carriers within the mucosal delivery methods and compositions of the invention. Two components are necessary for bacterial adherence processes, a bacterial ‘adhesin’ (adherence or colonization factor) and a receptor on the host cell surface. Bacteria causing mucosal infections need to penetrate the mucus layer before attaching themselves to the epithelial surface. This attachment is usually mediated by bacterial fimbriae or pilus structures, although other cell surface components may also take part in the process. Adherent bacteria colonize mucosal epithelia by multiplication and initiation of a series of biochemical reactions inside the target cell through signal transduction mechanisms (with or without the help of toxins). Associated with these invasive mechanisms, a wide diversity of bioadhesive proteins (e.g., invasin, internalin) originally produced by various bacteria and viruses are known. These allow for extracellular attachment of such microorganisms with an impressive selectivity for host species and even particular target tissues. Signals transmitted by such receptor-ligand interactions trigger the transport of intact, living microorganisms into, and eventually through, epithelial cells by endo- and transcytotic processes. Such naturally occurring phenomena may be harnessed (e.g., by complexing biologically active agents such as Y2 receptor-binding peptide with an adhesin) according to the teachings herein for enhanced delivery of biologically active compounds into or across mucosal epithelia and/or to other designated target sites of drug action.

Various bacterial and plant toxins that bind epithelial surfaces in a specific, lectin-like manner are also useful within the methods and compositions of the invention. For example, diphtheria toxin (DT) enters host cells rapidly by RME. Likewise, the B subunit of the E. coli heat labile toxin binds to the brush border of intestinal epithelial cells in a highly specific, lectin-like manner. Uptake of this toxin and transcytosis to the basolateral side of the enterocytes has been reported in vivo and in vitro. Other researches have expressed the transmembrane domain of diphtheria toxin in E. coli as a maltose-binding fusion protein and coupled it chemically to high-Mw poly-L-lysine. The resulting complex was successfully used to mediate internalization of a reporter gene in vitro. In addition to these examples, Staphylococcus aureus produces a set of proteins (e.g., staphylococcal enterotoxin A (SEA), SEB, toxic shock syndrome toxin 1 (TSST-1) which act both as superantigens and toxins. Studies relating to these proteins have reported dose-dependent, facilitated transcytosis of SEB and TSST-1 in Caco-2 cells.

Viral haemagglutinins comprise another type of transport agent to facilitate mucosal delivery of biologically active agents within the methods and compositions of the invention. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus. These and other exemplary viral hemagglutinins can be employed in a combinatorial formulation (e.g., a mixture or conjugate formulation) or coordinate administration protocol with one or more of the Y2 receptor-binding peptide, analogs and mimetics disclosed herein, to coordinately enhance mucosal delivery of one or more additional biologically active agent(s). Alternatively, viral hemagglutinins can be employed in a combinatorial formulation or coordinate administration protocol to directly enhance mucosal delivery of one or more of the Y2 receptor-binding peptide proteins, analogs and mimetics, with or without enhanced delivery of an additional biologically active agent.

A variety of endogenous, selective transport-mediating factors are also available for use within the invention. Mammalian cells have developed an assortment of mechanisms to facilitate the internalization of specific substrates and target these to defined compartments. Collectively, these processes of membrane deformations are termed ‘endocytosis’ and comprise phagocytosis, pinocytosis, receptor-mediated endocytosis (clathrin-mediated RME), and potocytosis (non-clathrin-mediated RME). RME is a highly specific cellular biologic process by which, as its name implies, various ligands bind to cell surface receptors and are subsequently internalized and trafficked within the cell. In many cells the process of endocytosis is so active that the entire membrane surface is internalized and replaced in less than a half hour. Two classes of receptors are proposed based on their orientation in the cell membrane; the amino terminus of Type I receptors is located on the extracellular side of the membrane, whereas Type II receptors have this same protein tail in the intracellular milieu.

Still other embodiments of the invention utilize transferrin as a carrier or stimulant of RME of mucosally delivered biologically active agents. Transferrin, an 80 kDa iron-transporting glycoprotein, is efficiently taken up into cells by RME. Transferrin receptors are found on the surface of most proliferating cells, in elevated numbers on erythroblasts and on many kinds of tumors. The transcytosis of transferrin (Tf) and transferrin conjugates is reportedly enhanced in the presence of Brefeldin A (BFA), a fungal metabolite. In other studies, BFA treatment has been reported to rapidly increase apical endocytosis of both ricin and HRP in MDCK cells. Thus, BFA and other agents that stimulate receptor-mediated transport can be employed within the methods of the invention as combinatorially formulated (e.g., conjugated) and/or coordinately administered agents to enhance receptor-mediated transport of biologically active agents, including Y2 receptor-binding peptide proteins, analogs and mimetics.

Polymeric Delivery Vehicles and Methods

Within certain aspects of the invention, Y2 receptor-binding peptide proteins, analogs and mimetics, other biologically active agents disclosed herein, and delivery-enhancing agents as described above, are, individually or combinatorially, incorporated within a mucosally (e.g., nasally) administered formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the Y2 receptor-binding peptide, analog or mimetic, can be functionalized in a manner where it can be covalently bound to the polymer and rendered inseparable from the polymer by simple washing. In other embodiments, the polymer is chemically modified with an inhibitor of enzymes or other agents which may degrade or inactivate the biologically active agent(s) and/or delivery enhancing agent(s). In certain formulations, the polymer is a partially or completely water insoluble but water swellable polymer, e.g., a hydrogel. Polymers useful in this aspect of the invention are desirably water interactive and/or hydrophilic in nature to absorb significant quantities of water, and they often form hydrogels when placed in contact with water or aqueous media for a period of time sufficient to reach equilibrium with water. In more detailed embodiments, the polymer is a hydrogel which, when placed in contact with excess water, absorbs at least two times its weight of water at equilibrium when exposed to water at room temperature, U.S. Pat. No. 6,004,583.

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Biodegradable polymers such as poly(glycolic acid) (PGA), poly-(lacetic acid) (PLA), and poly(D,L-lacetic-co-glycolic acid) (PLGA), have received considerable attention as possible drug delivery carriers, since the degradation products of these polymers have been found to have low toxicity. During the normal metabolic function of the body these polymers degrade into carbon dioxide and water. These polymers have also exhibited excellent biocompatibility.

For prolonging the biological activity of Y2 receptor-binding peptide, analogs and mimetics, and other biologically active agents disclosed herein, as well as optional delivery-enhancing agents, these agents may be incorporated into polymeric matrices, e.g., polyorthoesters, polyanhydrides, or polyesters. This yields sustained activity and release of the active agent(s), e.g., as determined by the degradation of the polymer matrix. Although the encapsulation of biotherapeutic molecules inside synthetic polymers may stabilize them during storage and delivery, the largest obstacle of polymer-based release technology is the activity loss of the therapeutic molecules during the formulation processes that often involve heat, sonication or organic solvents.

Absorption-promoting polymers contemplated for use within the invention may include derivatives and chemically or physically modified versions of the foregoing types of polymers, in addition to other naturally occurring or synthetic polymers, gums, resins, and other agents, as well as blends of these materials with each other or other polymers, so long as the alterations, modifications or blending do not adversely affect the desired properties, such as water absorption, hydrogel formation, and/or chemical stability for useful application. In more detailed aspects of the invention, polymers such as nylon, acrylan and other normally hydrophobic synthetic polymers may be sufficiently modified by reaction to become water swellable and/or form stable gels in aqueous media.

Absorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel”, Xanathan gums such as “Keltrol”, estastin, alpha hydroxy butyrate and its copolymers, hyaluronic acid and its derivatives, polylacetic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Other vinylidene monomers, including the acrylic nitriles, may also be used as absorption-promoting agents within the methods and compositions of the invention to enhance delivery and absorption of one or more Y2 receptor-binding peptide proteins, analogs and mimetics, and other biologically active agent(s), including to enhance delivery of the active agent(s) to a target tissue or compartment in the subject (e.g., the liver, hepatic portal vein, CNS tissue or fluid, or blood plasma). Useful alpha, beta-olefinically unsaturated nitriles are preferably monoolefinically unsaturated nitriles having from 3 to 10 carbon atoms such as acrylonitrile, methacrylonitrile, and the like. Most preferred are acrylonitrile and methacrylonitrile. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like.

Yet additional useful absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis (beta-haloalkyl) alkenyl phosphonates such as bis(beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

When hydrogels are employed as absorption promoting agents within the invention, these may be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Specific illustrative examples of useful polymers, especially for the delivery of peptides or proteins, are the following types of polymers: (meth)acrylamide and 0.1 to 99 wt. % (meth)acrylic acid; (meth)acrylamides and 0.1-75 wt % (meth)acryloxyethyl trimethyammonium chloride; (meth)acrylamide and 0.1-75 wt % (meth)acrylamide; acrylic acid and 0.1-75 wt % alkyl(meth)acrylates; (meth)acrylamide and 0.1-75 wt % AMPS® (trademark of Lubrizol Corp.); (meth)acrylamide and 0 to 30 wt % alkyl(meth)acrylamides and 0.1-75 wt % AMPS®; (meth)acrylamide and 0.1-99 wt. % HEMA; (metb)acrylamide and 0.1 to 75 wt % HEMA and 0.1 to 99% (meth)acrylic acid; (meth)acrylic acid and 0.1-99 wt % HEMA; 50 mole % vinyl ether and 50 mole % maleic anhydride; (meth)acrylamide and 0.1 to 75 wt % (meth)acryloxyalky dimethyl benzylammonium chloride; (meth)acrylamide and 0.1 to 99 wt % vinyl pyrrolidone; (meth)acrylamide and 50 wt % vinyl pyrrolidone and 0.1-99.9 wt % (meth)acrylic acid; (meth)acrylic acid and 0.1 to 75 wt % AMPS® and 0.1-75 wt % alkyl(meth)acrylamide. In the above examples, alkyl means C₁ to C₃₀, preferably C₁ to C₂₂, linear and branched and C₄ to C₁₆ cyclic; where (meth) is used, it means that the monomers with and without the methyl group are included. Other very useful hydrogel polymers are swellable, but insoluble versions of poly(vinyl pyrrolidone) starch, carboxymethyl cellulose and polyvinyl alcohol.

Additional polymeric hydrogel materials useful within the invention include (poly hydroxyalkyl(meth)acrylate: anionic and cationic hydrogels: poly(electrolyte) complexes; poly(vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

Other gelable, fluid imbibing and retaining polymers useful for forming the hydrophilic hydrogel for mucosal delivery of biologically active agents within the invention include pectin; polysaccharides such as agar, acacia, karaya, tragacenth, algins and guar and their crosslinked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of crosslinked poly(vinyl alcohol) and poly(N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides.

Synthetic hydrogel polymers for use within the invention may be made by an infinite combination of several monomers in several ratios. The hydrogel can be crosslinked and generally possesses the ability to imbibe and absorb fluid and swell or expand to an enlarged equilibrium state. The hydrogel typically swells or expands upon delivery to the nasal mucosal surface, absorbing about 2-5, 5-10, 10-50, up to 50-100 or more times fold its weight of water. The optimum degree of swellability for a given hydrogel will be determined for different biologically active agents depending upon such factors as molecular weight, size, solubility and diffusion characteristics of the active agent carried by or entrapped or encapsulated within the polymer, and the specific spacing and cooperative chain motion associated with each individual polymer.

Hydrophilic polymers useful within the invention are water insoluble but water swellable. Such water-swollen polymers as typically referred to as hydrogels or gels. Such gels may be conveniently produced from water-soluble polymer by the process of crosslinking the polymers by a suitable crosslinking agent. However, stable hydrogels may also be formed from specific polymers under defined conditions of pH, temperature and/or ionic concentration, according to know methods in the art. Typically the polymers are cross-linked, that is, cross-linked to the extent that the polymers possess good hydrophilic properties, have improved physical integrity (as compared to non cross-linked polymers of the same or similar type) and exhibit improved ability to retain within the gel network both the biologically active agent of interest and additional compounds for coadministration therewith such as a cytokine or enzyme inhibitor, while retaining the ability to release the active agent(s) at the appropriate location and time.

Generally hydrogel polymers for use within the invention are crosslinked with a difunctional cross-linking in the amount of from 0.01 to 25 weight percent, based on the weight of the monomers forming the copolymer, and more preferably from 0.1 to 20 weight percent and more often from 0.1 to 15 weight percent of the crosslinking agent. Another useful amount of a crosslinking agent is 0.1 to 10 weight percent. Tri, tetra or higher multifunctional crosslinking agents may also be employed. When such reagents are utilized, lower amounts may be required to attain equivalent crosslinking density, i.e., the degree of crosslinking, or network properties that are sufficient to contain effectively the biologically active agent(s).

The crosslinks can be covalent, ionic or hydrogen bonds with the polymer possessing the ability to swell in the presence of water containing fluids. Such crosslinkers and crosslinking reactions are known to those skilled in the art and in many cases are dependent upon the polymer system. Thus a crosslinked network may be formed by free radical copolymerization of unsaturated monomers. Polymeric hydrogels may also be formed by crosslinking preformed polymers by reacting functional groups found on the polymers such as alcohols, acids, amines with such groups as glyoxal, formaldehyde or glutaraldehyde, bis anhydrides and the like.

The polymers also may be cross-linked with any polyene, e.g. decadiene or trivinyl cyclohexane; acrylamides, such as N,N-methylene-bis(acrylamide); polyfunctional acrylates, such as trimethylol propane triacrylate; or polyfunctional vinylidene monomer containing at least 2 terminal CH₂<groups, including, for example, divinyl benzene, divinyl naphthlene, allyl acrylates and the like. In certain embodiments, cross-linking monomers for use in preparing the copolymers are polyalkenyl polyethers having more than one alkenyl ether grouping per molecule, which may optionally possess alkenyl groups in which an olefinic double bond is present attached to a terminal methylene grouping (e.g., made by the etherification of a polyhydric alcohol containing at least 2 carbon atoms and at least 2 hydroxyl groups). Compounds of this class may be produced by reacting an alkenyl halide, such as allyl chloride or allyl bromide, with a strongly alkaline aqueous solution of one or more polyhydric alcohols. The product may be a complex mixture of polyethers with varying numbers of ether groups. Efficiency of the polyether cross-linking agent increases with the number of potentially polymerizable groups on the molecule. Typically, polyethers containing an average of two or more alkenyl ether groupings per molecule are used. Other cross-linking monomers include for example, diallyl esters, dimethallyl ethers, allyl or methallyl acrylates and acrylamides, tetravinyl silane, polyalkenyl methanes, diacrylates, and dimethacrylates, divinyl compounds such as divinyl benzene, polyallyl phosphate, diallyloxy compounds and phosphite esters and the like. Typical agents are allyl pentaerythritol, allyl sucrose, trimethylolpropane triacrylate, 1,6-hexanediol diacrylate, trimethylolpropane diallyl ether, pentaerythritol triacrylate, tetramethylene dimethacrylate, ethylene diacrylate, ethylene dimethacrylate, triethylene glycol dimethacrylate, and the like. Allyl pentaerythritol and trimethylolpropane diallylether provide suitable polymers. When the cross-linking agent is present, the polymeric mixtures usually contain between about 0.01 to 20 weight percent, e.g., 1%, 5%, or 10% or more by weight of cross-linking monomer based on the total of carboxylic acid monomer, plus other monomers.

In more detailed aspects of the invention, mucosal delivery of Y2 receptor-binding peptide, analogs and mimetics, and other biologically active agents disclosed herein, is enhanced by retaining the active agent(s) in a slow-release or enzymatically or physiologically protective carrier or vehicle, for example a hydrogel that shields the active agent from the action of the degradative enzymes. In certain embodiments, the active agent is bound by chemical means to the carrier or vehicle, to which may also be admixed or bound additional agents such as enzyme inhibitors, cytokines, etc. The active agent may alternately be immobilized through sufficient physical entrapment within the carrier or vehicle, e.g., a polymer matrix.

Storage of Material

In certain aspects of the invention, the combinatorial formulations and/or coordinate administration methods herein incorporate an effective amount of peptides and proteins which may adhere to charged glass thereby reducing the effective concentration in the container. Silanized containers, for example, silanized glass containers, are used to store the finished product to reduce adsorption of the polypeptide or protein to a glass container.

In yet additional aspects of the invention, a kit for treatment of a mammalian subject comprises a stable pharmaceutical composition of one or more Y2 receptor-binding peptide compound(s) formulated for mucosal delivery to the mammalian subject wherein the composition is effective to alleviate one or more symptom(s) of obesity, cancer, or malnutrition or wasting related to cancer in said subject without unacceptable adverse side effects. The kit further comprises a pharmaceutical reagent vial to contain the one or more Y2 receptor-binding peptide compounds. The pharmaceutical reagent vial is composed of pharmaceutical grade polymer, glass or other suitable material. The pharmaceutical reagent vial is, for example, a silanized glass vial. The kit further comprises an aperture for delivery of the composition to a nasal mucosal surface of the subject. The delivery aperture is composed of a pharmaceutical grade polymer, glass or other suitable material. The delivery aperture is, for example, a silanized glass.

A silanization technique combines a special cleaning technique for the surfaces to be silanized with a silanization process at low pressure. The silane is in the gas phase and at an enhanced temperature of the surfaces to be silanized. The method provides reproducible surfaces with stable, homogeneous and functional silane layers having characteristics of a monolayer. The silanized surfaces prevent binding to the glass of polypeptides or mucosal delivery enhancing agents of the present invention.

The procedure is useful to prepare silanized pharmaceutical reagent vials to hold Y2 receptor-binding peptide compositions of the present invention. Glass trays are cleaned by rinsing with double distilled water (ddH₂O) before using. The silane tray is then be rinsed with 95% EtOH, and the acetone tray is rinsed with acetone. Pharmaceutical reagent vials are sonicated in acetone for 10 minutes. After the acetone sonication, reagent vials are washed in ddH₂O tray at least twice. Reagent vials are sonicated in 0.1M NaOH for 10 minutes. While the reagent vials are sonicating in NaOH, the silane solution is made under a hood. (Silane solution: 800 mL of 95% ethanol; 96 L of glacial acetic acid; 25 mL of glycidoxypropyltrimethoxy silane). After the NaOH sonication, reagent vials are washed in ddH₂O tray at least twice. The reagent vials are sonicated in silane solution for 3 to 5 minutes. The reagent vials are washed in 100% EtOH tray. The reagent vials are dried with prepurified N₂ gas and stored in a 100° C. oven for at least 2 hours before using.

Formulation and Administration

Mucosal delivery formulations of the present invention comprise Y2 receptor-binding peptide, analogs and mimetics, typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Within the compositions and methods of the invention, the Y2 receptor-binding peptide proteins, analogs and mimetics, and other biologically active agents disclosed herein may be administered to subjects by a variety of mucosal administration modes, including by oral, rectal, vaginal, intranasal, intrapulmonary, or transdermal delivery, or by topical delivery to the eyes, ears, skin or other mucosal surfaces. Optionally, Y2 receptor-binding peptide proteins, analogs and mimetics, and other biologically active agents disclosed herein can be coordinately or adjunctively administered by non-mucosal routes, including by intramuscular, subcutaneous, intravenous, intra-atrial, intra-articular, intraperitoneal, or parenteral routes. In other alternative embodiments, the biologically active agent(s) can be administered ex vivo by direct exposure to cells, tissues or organs originating from a mammalian subject, for example as a component of an ex vivo tissue or organ treatment formulation that contains the biologically active agent in a suitable, liquid or solid carrier.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 4.5±0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5μ mass median equivalent aerodynamic diameter (MMEAD), commonly about 1μ MMEAD, and more typically about 2μ MMEAD. Maximum particle size appropriate for deposition within the nasal passages is often about 10μ MMEAD, commonly about 8μ MMEAD, and more typically about 4μ MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which rely on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylacetic acid, poly(lacetic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, magnesium carbonate, and the like.

Therapeutic compositions for administering the biologically active agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants.

In certain embodiments of the invention, the biologically active agent is administered in a time-release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin. When controlled release formulations of the biologically active agent is desired, controlled release binders suitable for use in accordance with the invention include any biocompatible controlled-release material which is inert to the active agent and which is capable of incorporating the biologically active agent. Numerous such materials are known in the art. Useful controlled-release binders are materials that are metabolized slowly under physiological conditions following their intranasal delivery (e.g., at the nasal mucosal surface, or in the presence of bodily fluids following transmucosal delivery). Appropriate binders include but are not limited to biocompatible polymers and copolymers previously used in the art in sustained release formulations. Such biocompatible compounds are non-toxic and inert to surrounding tissues, and do not trigger significant adverse side effects such as nasal irritation, immune response, inflammation, or the like. They are metabolized into metabolic products that are also biocompatible and easily eliminated from the body.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

Mucosal administration according to the invention allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. In one embodiment, metered doses of active agent are delivered by means of a specially constructed mechanical pump valve, U.S. Pat. No. 4,511,069.

Dosage

For prophylacetic and treatment purposes, the biologically active agent(s) disclosed herein may be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). In this context, a therapeutically effective dosage of the Y2 receptor-binding peptide may include repeated doses within a prolonged prophylaxis or treatment regimen that will yield clinically significant results to alleviate one or more symptoms or detectable conditions associated with a targeted disease or condition as set forth above. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

The actual dosage of biologically active agents will of course vary according to factors such as the disease indication and particular status of the subject (e.g., the subject's age, size, fitness, extent of symptoms, susceptibility factors, etc), time and route of administration, other drugs or treatments being administered concurrently, as well as the specific pharmacology of the biologically active agent(s) for eliciting the desired activity or biological response in the subject. Dosage regimens may be adjusted to provide an optimum prophylacetic or therapeutic response. A therapeutically effective amount is also one in which any toxic or detrimental side effects of the biologically active agent are outweighed in clinical terms by therapeutically beneficial effects. A non-limiting range for a therapeutically effective amount of an Y2 agonist within the methods and formulations of the invention is 0.7 μg/kg to about 25 μg/kg. To promote weight loss, an intranasal dose of Y2 receptor-binding peptide is administered at dose high enough to promote satiety but low enough so as not to induce any unwanted side-effects such as nausea. A preferred intranasal dose of PYY₃₋₃₆ is about 1 μg-10 μg/kg weight of the patient, most preferably from about 1.5 μg/kg to about 3 μg/kg weight of the patient. In a standard dose a patient will receive 40 μg to 200 μg, more preferably about between 50 μg to 150 μg, most preferably 100 μg. Alternatively, a non-limiting range for a therapeutically effective amount of a biologically active agent within the methods and formulations of the invention is between about 0.001 pmol to about 100 pmol per kg body weight, between about 0.01 pmol to about 100 pmol per kg body weight, between about 0.1 pmol to about 5 pmol per kg body weight, or between about 0.5 pmol to about 1.0 pmol per kg body weight. Dosages within this range can be achieved by single or multiple administrations, including, e.g., multiple administrations per day, daily or weekly administrations. Repeated intranasal dosing with the formulations of the invention, on a schedule ranging from about 0.1 to 24 hours between doses, preferably between 0.5 and 24.0 hours between doses, will maintain normalized, sustained therapeutic levels of Y2 receptor-binding peptide to maximize clinical benefits while minimizing the risks of excessive exposure and side effects. This dose can be administered several times a day to promote satiety, preferably one half hour before a meal or when hunger occurs. The goal is to mucosally deliver an amount of the Y2 receptor-binding peptide sufficient to raise the concentration of the Y2 receptor-binding peptide in the plasma of an individual to mimic the concentration that would normally occur postprandially, i.e., after the individual has finished eating.

Dosage of Y2 agonists such as PYY may be varied by the attending clinician or patient, if self administering an over the counter dosage form, to maintain a desired concentration at the target site.

In an alternative embodiment, the invention provides compositions and methods for intranasal delivery of Y2 receptor-binding peptide, wherein the Y2 receptor-binding peptide compound(s) is/are repeatedly administered through an intranasal effective dosage regimen that involves multiple administrations of the Y2 receptor-binding peptide to the subject during a daily or weekly schedule to maintain a therapeutically effective elevated and lowered pulsatile level of Y2 receptor-binding peptide during an extended dosing period. The compositions and method provide Y2 receptor-binding peptide compound(s) that are self-administered by the subject in a nasal formulation between one and six times daily to maintain a therapeutically effective elevated and lowered pulsatile level of Y2 receptor-binding peptide during an 8 hour to 24 hour extended dosing period.

Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more Y2 receptor-binding peptide proteins, analogs or mimetics, and/or other biologically active agents in combination with mucosal delivery enhancing agents disclosed herein formulated in a pharmaceutical preparation for mucosal delivery.

The intranasal formulations of the present invention can be administered using any spray bottle or syringe. An example of a nasal spray bottle is the, “Nasal Spray Pump w/Safety Clip, Pfeiffer SAP # 60548, which delivers a dose of 0.1 mL per squirt and has a diptube length of 36.05 mm. It can be purchased from Pfeiffer of America of Princeton, N.J. Intranasal doses of a Y2 receptor-binding peptide such as PYY can range from 0.1 μg/kg to about 1500 μg/kg. When administered in as an intranasal spray, it is preferable that the particle size of the spray are between 10-100 μm (microns) in size, preferably 20-100 μm in size.

To promote weight loss, an intranasal dose of a Y2 receptor-binding peptide PYY is administered at dose high enough to promote satiety but low enough so as not to induce any unwanted side-effects such as nausea. A preferred intranasal dose of a Y2 receptor-binding peptide such as PYY(3-36) is about 3 μg-10 μg/kg weight of the patient, most preferably about 6 μg/kg weight of the patient. In a standard dose a patient will receive 50 μg to 800 μg, more preferably about between 100 μg to 400 μg, most preferably 150 μg to about 200 μg. The a Y2 receptor-binding peptide such as PYY(3-36) is preferably administered at least ten minutes to one hour prior to eating to prevent nausea but no more than about twelve to twenty-four hours prior to eating. The patient is dosed at least once a day preferably before every meal until the patient has lost a desired amount of weight. The patient then receives maintenance doses at least once a week preferably daily to maintain the weight loss.”

As is shown by the data from the following examples, when administered intranasally to humans using the Y2 receptor-binding peptide formulation of the present invention, PYY(3-36) was found to reduce appetite. The examples also show that for the first time post-prandial physiological levels of a PYY peptide could be reached through an intranasal route of administration using the Y2 receptor-binding peptide formulations of the present invention in which PYY(3-36) was the Y2 receptor-binding peptide.

### Aerosol Nasal Administration of PYY

We have discovered that the Y2 receptor-binding peptides can be administered intranasally using a nasal spray or aerosol. This is surprising because many proteins and peptides have been shown to be sheared or denatured due to the mechanical forces generated by the actuator in producing the spray or aerosol. In this area the following definitions are useful.

1. Aerosol—A product that is packaged under pressure and contains therapeutically active ingredients that are released upon activation of an appropriate valve system.

2. Metered aerosol—A pressurized dosage form comprised of metered dose valves, which allow for the delivery of a uniform quantity of spray upon each activation.

3. Powder aerosol—A product that is packaged under pressure and contains therapeutically active ingredients in the form of a powder, which are released upon activation of an appropriate valve system.

4. Spray aerosol—An aerosol product that utilizes a compressed gas as the propellant to provide the force necessary to expel the product as a wet spray; it generally applicable to solutions of medicinal agents in aqueous solvents.

5. Spray—A liquid minutely divided as by a jet of air or steam. Nasal spray drug products contain therapeutically active ingredients dissolved or suspended in solutions or mixtures of excipients in nonpressurized dispensers.

6. Metered spray—A non-pressurized dosage form consisting of valves that allow the dispensing of a specified quantity of spray upon each activation.

7. Suspension spray—A liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

Thorough characterization of the spray's geometry has been found to be the best indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps.

Definitions

Plume Height—the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study

Major Axis—the largest chord that can be drawn within the fitted spray pattern that crosses the COMw in base units (mm)

Minor Axis—the smallest chord that can be drawn within the fitted spray pattern that crosses the COMw in base units (mm)

Ellipticity Ratio—the ratio of the major axis to the minor axis

D₁₀—the diameter of droplet for which 10% of the total liquid volume of sample consists of droplets of a smaller diameter (μm)

D₅₀—the diameter of droplet for which 50% of the total liquid volume of sample consists of droplets of a smaller diameter (μm), also known as the mass median diameter

D₉₀—the diameter of droplet for which 90% of the total liquid volume of sample consists of droplets of a smaller diameter (μm)

Span—measurement of the width of the distribution, The smaller the value, the narrower the distribution. Span is calculated as

$$\frac{( {D}_{90} - {D}_{10} )}{{D}_{50}} .$$

% RSD—percent relative standard deviation, the standard deviation divided by the mean of the series and multiplied by 100, also known as % CV.

The following examples are provided by way of illustration, not limitation.

### EXAMPLE 1

### Preparation of a PYY3-36 in the Presence of Me-b-CD, DDPC, EDTA, Citrate Buffer, Lactose, Sorbitol, and Chlorobutanol

A PYY formulation suitable for intranasal administration of PYY, which was substantially free of a stabilizer that is a protein was prepared having the formulation listed in Table 1 below. The primary tonicifiers in the formulation were lactose and sorbitol. Chlorobutanol was present as a preservative to allow for potential multi-use. Me-b-CD, DDPC and EDTA provided for permeation enhancement.
1. About ¾ of the water was added to a beaker and stirred with a stir bar on a stir plate and the sodium citrate was added until it was completely dissolved.
2. The EDTA was then added and stirred until it was completely dissolved.
3. The citric acid was then added and stirred until it was completely dissolved.
4. The methyl-β-cyclodextrin was added and stirred until it was completely dissolved.
5. The DDPC was then added and stirred until it was completely dissolved.
6. The lactose was then added and stirred until it was completely dissolved.
7. The sorbitol was then added and stirred until it was completely dissolved.
8. The chlorobutanol was then added and stirred until it was completely dissolved.
9. The PYY 3-36 was added and stirred gently until it dissolved.
10. 11 Check the pH to make sure it is 5.0±0.25. Add dilute HCl or dilute NaOH to adjust the pH.

11. Add water to final volume.

TABLE 1
Preparation of a PYY3-36 in the Presence of Me-b-CD, DDPC,
EDTA, Citrate Buffer, Lactose, Sorbitol, and Chlorobutanol

| Reagent | mg/mL | % |
|---|---|---|
| Chlorobutanol, anhydrous | 5.0 | 0.50 |
| Methyl-β-Cyclodextrin | 45 | 4.5 |
| L-α-Phosphatidylcholine Didecanoyl | 1 | 0.1 |
| Edetate Disodium | 1 | 0.1 |
| Sodium Citrate, Dihydrate | 1.62 | 0.162 |
| Citric Acid, Anhydrous | 0.86 | 0.086 |
| α-Lactose monohydrate | 9 | 0.9 |
| Sorbitol | 18.2 | 1.82 |
| PYY 3-36 | 1 or 15 | 0.1 |
| Water | | qs |

Measurement of Osmolarity

Formulation samples were measured by Model 2020 from Advanced Instruments Inc. (Norwood, Mass.). The sample produced as described in the above Table 1 resulted in a pH of 5.0 and an osmolarity of 225 mOsm.

### EXAMPLE 2

### In Vitro Performance Comparison of Endotoxin-Free Vs. Non-Endotoxin-Free PYY₃₋₃₆

A study was conducted comparing the ability of endotoxin-free PYY(3-36) (SEQ ID NO: 2) vs. non-endotoxin-free PYY(3-36) to permeate across a mucosal tissue barrier in vitro. The experimental procedures for the in vitro tissue studies are described below.

Cell Cultures

The cell line MatTek Corp. (Ashland, Mass.) was the source of normal, human-derived tracheal/bronchial epithelial cells (EpiAirway™ Tissue Model). The cells were provided as inserts grown to confluent on Millipore Milicell-CM filters comprised of transparent hydrophilic Teflon (PTFE). The membranes were cultured in 1 mL basal media (phenol red-free and hydrocortisone-free Dulbecco's Modified Eagle's Medium (DMEM)) at 37° C. with 5% CO₂ for 24-48 hours before use. Inserts were feed for each day of recovery. Same media was used during testing.

Tissue Permeation Assay

Each tissue insert was placed in an individual well containing 1 mL of basal media. On the apical surface of the inserts, 100 ul of test formulation was applied, and the samples were placed on a shaker (˜100 rpm) for 120 min at 37° C. The underlying culture media samples was stored at 2-8° C. until needed. Transepithelial electrical resistance (TEER) was measured before and after the 2-h incubation. Following the incubation, the cell inserts were analyzed for cell viability via the mitochondrial dehydrogenase (MDH) assay.

Transepithelial Electrical Resistance (TEER)

TEER measurements were read using a Tissue Resistance Measurement Chamber connected to an Epithelial Voltobmeter with the electrode leads, both from World Precision Instruments.

The electrodes and a tissue culture blank insert were equilibrated for at least 20 min in fresh media with the power off prior to checking calibration. The background resistance was measured with 1.5 ml media in the Endohm tissue chamber and 300 ul media in a blank Millicell-CM insert. The top electrode was adjusted so that is submerged in the media but not making contact with the top surface of the insert membrane. Background resistance of the blank insert was 5-20 ohms. For each TEER determination, 300 ul media was added to the insert followed by 20 min incubation at RT before placement in the Endohm chamber to read TEER. Resistance was expressed as (resistance measured−blank)×0.6 cm².

MTT Assay

Cell viability was assessed using the MTT assay kit, cat #MTT-100 by MatTek Corp. This kit measures the uptake and transformation of tetrazolium salt to its formazan dye (MTT).

MTT concentrate was thawed and diluted with media at a ratio of 2 ml MTT: 8 ml media. 300 ul MTT-media mix was added to each well in a fresh 24-well plate. Tissue culture inserts were gently drained on paper towel and transferred to the MTT containing well, and then incubated at 37 deg ° C. in the dark for 3 hours.

After incubation, each insert was removed from the plate, blotted gently (the bottom of the insert on a paper towel) and then immersed in wells of a fresh 24-well plate, each containing 2 ml extracting solution. The plate was then be covered to reduce evaporation and incubated overnight at RT in the dark.

The next day, the liquid in each insert was decanted back into the well from which it was taken. 50 μl of the extracting solution from each well was pipetted in triplicate into a 96 well plate and diluted with the addition of 150 ul of fresh extracting solution. Extract blanks were in triplicate. The optical density of the samples was measured at 550 nm on μQuant optical density plate reader using KCJr software.

LDH Assay

The amount of cell death will be assayed by measuring the loss of lactate dehydrogenase (LDH) from the cells using a CytoTox 96 Cytoxicity Assay Kit (Promega Corp., Madison, Wis.). Fifty microliters of sample will be loaded in triplicate into 96 well assay plates. Fresh, cell-free culture medium will be used as a blank. Fifty microliters of substrate solution will be added to each well and the plates incubated for 30 minutes at room temperature in the dark. Following incubation, 50 μL of stop solution will be added to each well and the plates read on an optical density plate reader at 490 nm.

Enzyme Immunoassay (EIA)

The % permeation of PYY ₃₋₃₆ that cross from the apical side to the basal side during the 1 hour incubation period was assayed with a PYY ₃₋₃₆ EIA kits purchased from Phoenix Pharmaceuticals, Inc. (Belmont, Calif.). Samples are diluted appropriate with assay buffer that is provided with the kit. The protocol that comes with the kits will be used.

It was determined that about twice the amount of endotoxin-free PYY(3-36) permeated the bronchial epithelium as compared to PYY(3-36) formulation that contained endotoxin.

Both formulations contained Chlorobutanol 2.5 mg/ml, 2. mg/ml of DDPC, 10 mg/ml of albumin, 1 mg/ml of EDTA (edetate disodium) and 45 mg/ml of M-B-CD. One formulation contained endotoxin-free PYY(3-36) and the other formulation contained 70 EUs or greater of endotoxin.

The average cell viability (MTT assay) of the PYY(3-36) formulation containing endotoxin was 91.72% while the endotoxin-free PYY(3-36) formulation had an average MTT of 100.16%. Data for cytotoxicity (LDH assay) were similar for endotoxin-free and non-endotoxin-free peptide.

The average permeation of the PYY(3-36) formulation containing endotoxin was 5.36%, while the average permeation of the endotoxin-free PYY(3-36) formulation was 10.75%.

A number of known mucosal delivery enhancing excipients can be effectively combined with endotoxin-free Y2 receptor binding peptides, especially endotoxin-free PYY3-36, and can be used to improve non-infusion formulations, especially oral delivery. Such excipients are contained in the following patent applications that are incorporated by reference: US Patent applications 20030225300, 20030198658, 20030133953, 20030078302, 20030045579, 20030012817, 20030012817, 20030008900, 20020155993, 20020127202, 20020120009, 20020119910, 20020065255, 20020052422, 20020040061, 20020028250, 20020013497, 20020001591, 20010039258, 20010003001.

### EXAMPLE 3

### Effect of Osmolarity on Stability Towards Thermal Stress for PYY₃₋₃₆ in the Presence of Me-b-CD, DDPC, EDTA, Citrate Buffer, Chlorobutanol and Either Sodium Chloride or Lactose/Sorbitol as Tonicifier

In this example, a series of samples were produced all having the same levels of Me-b-CD, DDPC, EDTA citrate buffer and chlorobutanol. In the series, the osmolarity was varied from 90 to 300 mOsm by varying the level of the tonicifiers sodium chloride. For comparison, the case is also shown where the combination of lactose and sorbital are present as tonicifiers with osmolarity of 225 mOsm. The samples are listed in Table 2 below:

TABLE 2
Samples Tested in Example 3.

| Sample | Composition | Comments |
|---|---|---|
| 1 | 1 mg/mL PYY, 45 mg/mL M-b-CD, 1 | pH 5.0 and |
| | mg/mL EDTA, 1 mg/mL DDPC, 10 mM | 225 mOsm |
| | citrate buffer pH 5.0, 25 mM lactose, | |
| | 100 mM sorbitol, 0.5% CB | |
| 2 | 1 mg/mL PYY, 45 mg/mL M-b-CD, 1 | pH 5.0 and |
| | mg/mL EDTA, 1 mg/mL DDPC, 10 mM | 90 mOsm |
| | citrate buffer pH 5.00, 0.5% CB | |
| 3 | 1 mg/mL PYY, 45 mg/mL M-b-CD, 1 | pH 5.0 and |
| | mg/mL EDTA, 1 mg/mL DDPC, 10 mM | 200 mOsm |
| | citrate buffer pH 5.00, 52.5 mM NaCl, | |
| | 0.5% CB | |
| 4 | 1 mg/mL PYY, 45 mg/mL M-b-CD, 1 | pH 5.0 and |
| | mg/mL EDTA, 1 mg/mL DDPC, 10 mM | 225 mOsm |
| | citrate buffer pH 5.00, 65.0 mM NaCl, | |
| | 0.5% CB | |
| 5 | 1 mg/mL PYY, 45 mg/mL M-b-CD, 1 | pH 5.0 and |
| | mg/mL EDTA, 1 mg/mL DDPC, 10 mM | 250 mOsm |
| | citrate buffer pH 5.00, 77.5 mM NaCl, | |
| | 0.5% CB | |
| 6 | 1 mg/mL PYY, 45 mg/mL M-b-CD, 1 | pH 5.0 and |
| | mg/mL EDTA, 1 mg/mL DDPC, 10 mM | 275 mOsm |
| | citrate buffer pH 5.00, 90.0 mM NaCl, | |
| | 0.5% CB | |
| 7 | 1 mg/mL PYY, 45 mg/mL M-b-CD, 1 | pH 5.0 and |
| | mg/mL EDTA, 1 mg/mL DDPC, 10 mM | 300 mOsm |
| | citrate buffer pH 5.00, 102.5 mM NaCl, | |
| | 0.5% CB | |

Formulations were manufactured and immediately stored in Type 1, U-Save, amber glass vials capped with O'Berk fine-thread trifoil caps. For the purpose of this example, vials were subjected to thermal stress by storage at 40° C. A least one vial of each formulation at each storage temperature is sacrificed for HPLC analyzed at each timepoint.

HPLC

Formulations active and preservative content was assay using HPLC. The HPLC method uses a 5-micron C18 column (Supelco, BIO Wide-pore, 250×4.6 mm) at 45° C. with mobile phase components of 0.1% trifluoroacetic acid (A) and 0.08% trifluoroacetic acid in acentonitrile (B) delivered isocratically at 27% A/73% B. Detection was by UV at 210 nm. Quantitation was carried out by external standard method.

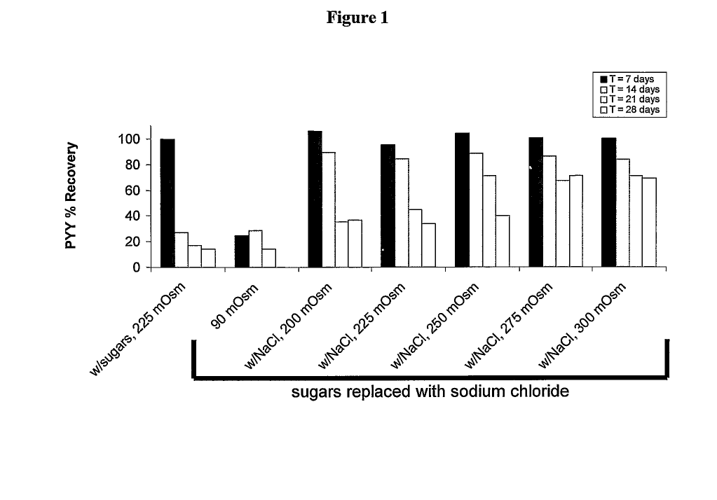

FIG. 1 shows the effect of osmolarity on stability towards thermal stress for PYY₃₋₃₆ in the presence of Me-b-CD, DDPC, EDTA, citrate buffer, sodium chloride, and chlorobutanol. As can be seen, stability in the presence of any tonicifer was greater than the case where no tonicifiers was present (90 mOsm). The best stability for the case of sodium chloride as tonicifiers was 250-300 mOsm.

### EXAMPLE 4

### Comparative Stability Towards Thermal Stress for PYY₃₋₃₆ in the Presence of Me-b-CD, DDPC, EDTA, Citrate Buffer, Lactose, Sorbitol and Either Chlorobutanol or Sodium Benzoate

Samples for this example are listed in Table 3. Samples were manufactured and stored in the same fashion as described in Example 3.

TABLE 3
Samples Tested in Example 4

| Sample | Composition | Comments |
|---|---|---|
| 1 | 1 mg/mL PYY, 45 mg/mL M-b-CD, 1 mg/mL | pH 5.0 and |
| | EDTA, 1 mg/mL DDPC, 10 mM citrate | 225 mOsm |
| | buffer pH 5.0, 25 mM lactose, 100 mM | |
| | sorbitol, 0.5% CB | |
| 2 | 1 mg/mL PYY, 45 mg/mL M-b-CD, 1 mg/mL | pH 4.3 and |
| | EDTA, 1 mg/mL DDPC, 10 mM citrate | 225 mOsm |
| | buffer pH 4.3, 25 mM lactose, 100 mM | |
| | sorbitol, 0.5% CB | |
| 3 | 1 mg/mL PYY, 45 mg/mL M-b-CD, 1 mg/mL | pH 5.0 and |
| | EDTA, 1 mg/mL DDPC, 10 mM citrate | 225 mOsm |
| | buffer pH 5.0, 65.0 mM NaCl, 0.5% CB | |
| 4 | 1 mg/mL PYY, 45 mg/mL M-b-CD, 1 mg/mL | pH 4.3 and |
| | EDTA, 1 mg/mL DDPC, 10 mM citrate | 225 mOsm |
| | buffer pH 4.3, 65.0 mM NaCl, 0.5% CB | |
| 5 | 1 mg/mL PYY, 45 mg/mL M-b-CD, 1 mg/mL | 0.50% Na |
| | EDTA, 1 mg/mL DDPC, 10 mM citrate | Benz; pH |
| | buffer pH 4.3, 25 mM lactose, 100 mM | 4.3 |
| | sorbitol, 0.50% Na Benz | |
| 6 | 1 mg/mL PYY, 45 mg/mL M-b-CD, 1 mg/mL | No sugars, |
| | EDTA, 1 mg/mL DDPC, 10 mM citrate | 0.50% Na |
| | buffer pH 4.3, 70 mM NaCl, 0.50% Na | Benz; pH 4.3 |
| | Benz | |

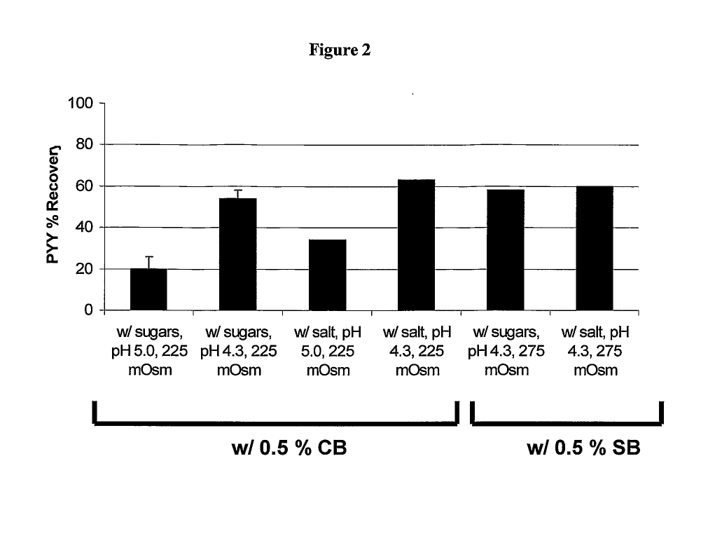

FIG. 2 depicts the data for stability of the various formulations tested in this Example 4. It can be seen that a pH of 4.3 provided superior stability (improved recovery of peptide over time) compared to the case of pH 5.0. The improved stability at the lower pH was observed for the case when sugar was used as tonicifiers as well as the case where salt was used as tonicifier.

### EXAMPLE 5

### Effect of Stability Towards Thermal Stress for PYY₃₋₃₆ in the Presence of Me-b-CD, DDPC, EDTA, Citrate Buffer, Chlorobutanol and either Lactose, Sorbitol and/or Sodium Chloride

The samples for this example are listed in Table 4. The samples were prepared and tested as described in Example 3.

TABLE 4
Samples Tested in Example 5

| Sample | Composition | Comments |
|---|---|---|
| 1 | 1 mg/mL PYY, 45 mg/mL M-b-CD, 1 mg/mL | pH 4.3 and |
| | EDTA, 1 mg/mL DDPC, 10 mM citrate | 225 mOsm |
| | buffer pH 4.3, 25 mM lactose, 100 mM | |
| | sorbitol, 0.5% CB | |
| 2 | 1 mg/mL PYY, 45 mg/mL M-b-CD, 1 mg/mL | No lactose, |
| | EDTA, 1 mg/mL DDPC, 10 mM citrate | 0.5% CB; |
| | buffer pH 4.3, 100 mM sorbitol, 0.5% CB | pH 4.3 |
| | | 200 mOsm |
| 3 | 1 mg/mL PYY, 45 mg/mL M-b-CD, 1 mg/mL | No lactose, |
| | EDTA, 1 mg/mL DDPC, 10 mM citrate | more sorbitol |
| | buffer pH 4.3, 125 mM sorbitol, 0.5% CB | 0.5% CB; |
| | | pH 4.3 and |
| | | 225 mOsm |
| 4 | 1 mg/mL PYY, 45 mg/mL M-b-CD, 1 mg/mL | No lactose, |
| | EDTA, 1 mg/mL DDPC, 10 mM citrate | 0.5% CB; |
| | buffer pH 4.3, 100 mM sorbitol, 12.5 | pH 4.3 and |
| | NaCl, 0.5% CB | 225 mOsm |
| 5 | 1 mg/mL PYY, 45 mg/mL M-b-CD, 1 mg/mL | pH 4.3 and |
| | EDTA, 1 mg/mL DDPC, 10 mM citrate | 225 mOsm |
| | buffer pH 4.3, 65.0 mM NaCl, 0.5% CB | |

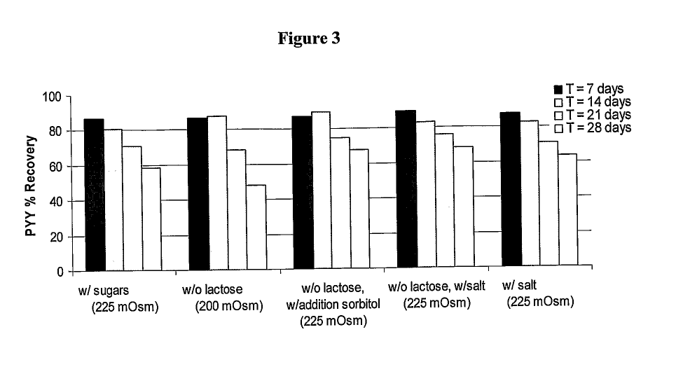

The stability data are depicted in FIG. 3. As long as the overall tonicity was 225 mOsm, stability was improved compared to the case where lactose was removed and the osmolarity was 200 mOsm. For example, when the osmolarity was 225 mOsm as provided by combination of lactose and sorbitol, or sorbitol alone, sorbitol and salt (sodium chloride) or salt (sodium chloride) alone there was superior stability compared to the case where the osmolarity was 200 mOsm (as provided by sorbitol alone).

### EXAMPLE 6

### Effect of pH on Stability Towards Thermal Stress for PYY₃₋₃₆ in the Presence of Me-b-CD, DDPC, EDTA, Citrate Buffer, Chlorobutanol and Sodium Chloride as the Tonicifer

Table 5 lists various samples produced where the pH was varied from 3.8 to 5.0 but otherwise containing the same levels of PYY₃₋₃₆, Me-b-CD, DDPC, EDTA, citrate buffer, chlorobutanol and sodium chloride. Samples were produced and tested under thermal stress as described in Example 3.

TABLE 5
Samples Tested in Example 6

| Sample | Composition | Comments |
|---|---|---|
| 1 | 1 mg/mL PYY, 45 mg/mL M-b-CD, 1 mg/mL | pH 3.8 and |
| | EDTA, 1 mg/mL DDPC, 10 mM citrate | 225 mOsm |
| | buffer pH 3.8, 65.0 mM NaCl, 0.5% CB | |
| 2 | 1 mg/mL PYY, 45 mg/mL M-b-CD, 1 mg/mL | pH 4.1 and |
| | EDTA, 1 mg/mL DDPC, 10 mM citrate | 225 mOsm |
| | buffer pH 4.1, 65.0 mM NaCl, 0.5% CB | |
| 3 | 1 mg/mL PYY, 45 mg/mL M-b-CD, 1 mg/mL | pH 4.3 and |
| | EDTA, 1 mg/mL DDPC, 10 mM citrate | 225 mOsm |
| | buffer pH 4.3, 65.0 mM NaCl, 0.5% CB | |
| 4 | 1 mg/mL PYY, 45 mg/mL M-b-CD, 1 mg/mL | pH 4.5 and |
| | EDTA, 1 mg/mL DDPC, 10 mM citrate | 225 mOsm |
| | buffer pH 4.5, 65.0 mM NaCl, 0.5% CB | |
| 5 | 1 mg/mL PYY, 45 mg/mL M-b-CD, 1 mg/mL | pH 4.8 and |
| | EDTA, 1 mg/mL DDPC, 10 mM citrate | 225 mOsm |
| | buffer pH 4.8, 65.0 mM NaCl, 0.5% CB | |
| 6 | 1 mg/mL PYY, 45 mg/mL M-b-CD, 1 mg/mL | pH 5.0 and |
| | EDTA, 1 mg/mL DDPC, 10 mM citrate | 225 mOsm |
| | buffer pH 5.0, 65.0 mM NaCl, 0.5% CB | |

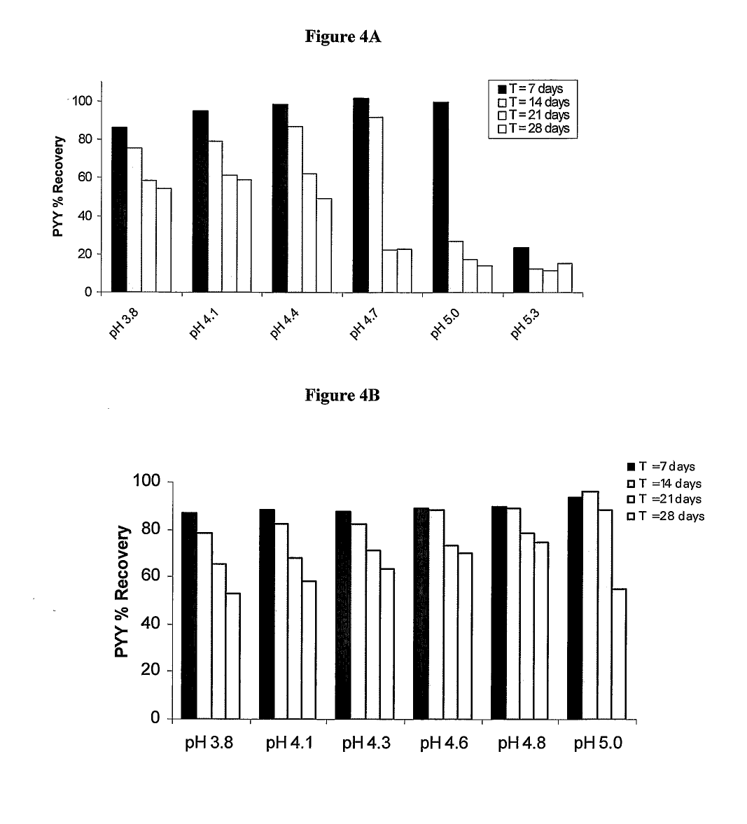

FIG. 4A depicts the data for stability of the formulation described in Table 5. The data show that the greatest instability was observed at the low and high limits over the pH range tested. For instance, at pH 3.8 and also at pH 5.0, the greatest loss of peptide after 28 days at 40 C was observed. The data suggest the preferred pH range for maintaining optimal stability to be 4.1 to 4.8.

### EXAMPLE 7

### Effect of Preservative on Stability Towards Thermal Stress for PYY₃₋₃₆ in the Presence of Me-b-CD, DDPC, EDTA, Citrate Buffer, Lactose and Sorbitol

Table 6 lists various samples produced where the preservative was varied from chlorobutanol to sodium benzoate but otherwise containing the same levels of PYY₃₋₃₆, Me-b-CD, DDPC, EDTA, citrate buffer, sorbitol and lactose. Samples were produced and tested under thermal stress as described in Example 3.

TABLE 6
Samples Tested in Example 7

| Sample | Composition | Comments |
|---|---|---|
| 1 | 1 mg/mL PYY, 45 mg/mL M-b-CD, 1 mg/mL | pH 4.3 and |
| | EDTA, 1 mg/mL DDPC, 10 mM citrate | 225 mOsm |
| | buffer pH 4.3, 25 mM lactose, 100 mM | |
| | sorbitol, 0.5% CB | |
| 2 | 1 mg/mL PYY, 45 mg/mL M-b-CD, 1 mg/mL | 0.50% Na |
| | EDTA, 1 mg/mL DDPC, 10 mM citrate | Benz instead |
| | buffer pH 4.3, 25 mM lactose, 100 mM | of 0.5% CB; |
| | sorbitol, 0.50% Na Benz | pH 4.3 |

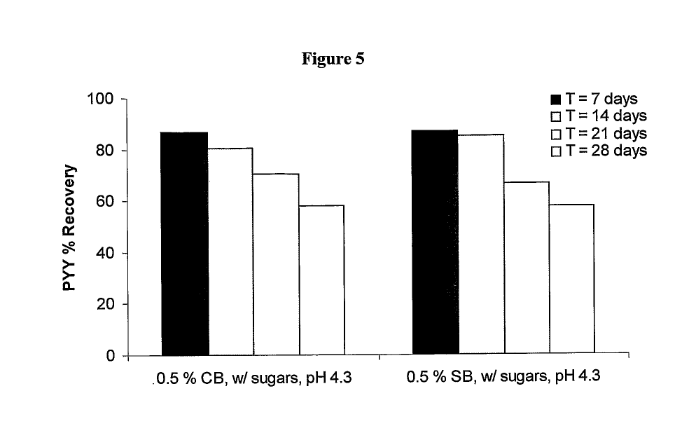

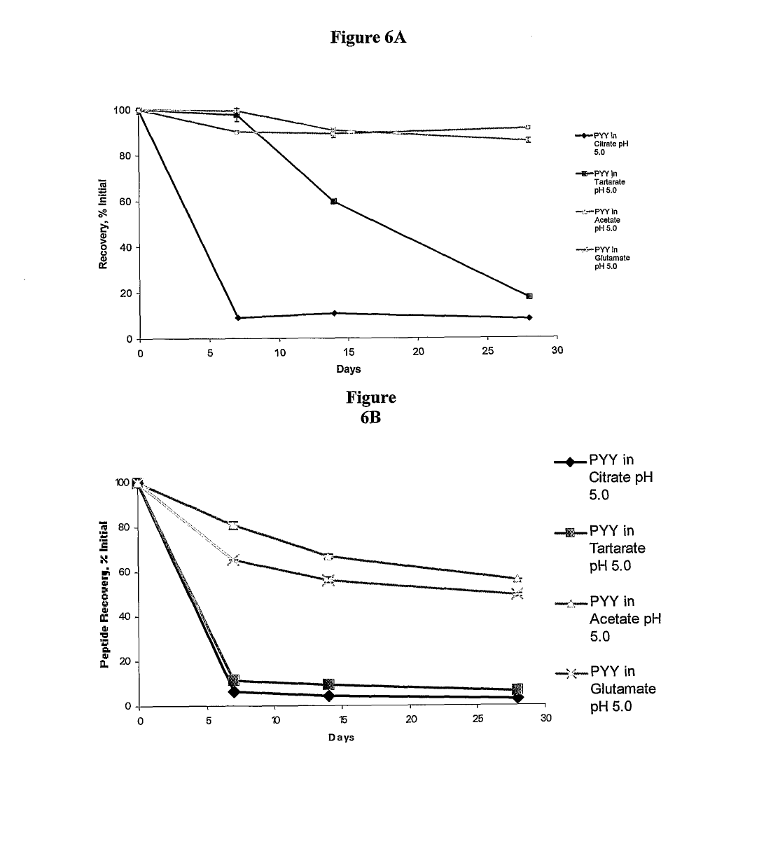

FIG. 5 shows no different in stability. Therefore, change in preservative from chlorobutanol to sodium benzoate does not affect PYY3-36 stability over the conditions tested.

### EXAMPLE 8

### Effect of pH Upon PYY₃₋₃₆ Under Thermal Stress

Table 7 lists various samples produced where the pH was varied from 3.8 to 5.0 but otherwise containing the same levels of PYY₃₋₃₆, Me-b-CD, DDPC, EDTA, citrate buffer, sorbitol, lactose, and chlorobutanol. Samples were produced and tested under thermal stress as described in Example 3.

TABLE 7
Samples Tested in Example 8.

| | | | | Citrate | | | | |
|---|---|---|---|---|---|---|---|---|
| Group | PYY | DDPC | EDTA | Buffer | Lactose | Sorbitol | CB | |
| # | (mg/mL) | (mg/mL) | (mg/mL) | (mM) | (mM) | (mM) | (mg/mL) | pH |
| 1 | 1 | 1 | 1 | 10 | 25 | 100 | 5 | 3.8 |
| 2 | 1 | 1 | 1 | 10 | 25 | 100 | 5 | 4.1 |
| 3 | 1 | 1 | 1 | 10 | 25 | 100 | 5 | 4.3 |
| 4 | 1 | 1 | 1 | 10 | 25 | 100 | 5 | 4.7 |
| 5 | 1 | 1 | 1 | 10 | 25 | 100 | 5 | 5.0 |
| 6 | 1 | 1 | 1 | 10 | 25 | 100 | 5 | 5.3 |
| 7 | 1 | 1 | 1 | 10 | 25 | 100 | 5 | 5.0 |

These data demonstrate that there is a pH-dependence to PYY₃₋₃₆ stability. Optimal stability is achieved at pH 3.8-4.4 under the conditions tested, as illustrated in FIG. 4B. Therefore, when sorbitol and lactose are used as the tonicifiers system, the preferred pH range to provide PYY₃₋₃₆ stability is between pH 3.8 and 4.7.

### EXAMPLE 9

### Effects of pH and Varying Tonicifiers Upon PYY₃₋₃₆ Nasal Spray In Vitro Performance

Formulations were manufactured as listed in Table 8. The formulations were then filled into 1-cc amber glass non-silanized vials, 0.5 mL fill per vial, and were fitted with a trifoil-lined polypropylene cap. All in vitro tissue model tested was conducted as described in Example 2.

TABLE 8
Samples Tested in Example 9

| | PYY | | | Citrate | | | Sodium | | | | |
|---|---|---|---|---|---|---|---|---|---|---|---|
| Group | (mg/ | DDPC | EDTA | Buffer | Lactose | Sorbitol | Chrloide | Trehalose | CB | | Osmolarity |
| # | mL) | (mg/mL) | (mg/mL) | (mM) | (mM) | (mM) | (mM) | (mM) | (mg/mL) | pH | (mOsm/kg) |
| 1 | 1 | 1 | 1 | 10 | 25 | 100 | 0 | 0 | 5 | 3.8 | 225 |
| 2 | 1 | 1 | 1 | 10 | 25 | 100 | 0 | 0 | 5 | 5.0 | 225 |
| 3 | 1 | 1 | 1 | 10 | 25 | 100 | 0 | 0 | 5 | 5.3 | 225 |
| 4 | 1 | 1 | 1 | 10 | 25 | 100 | 0 | 0 | 5 | 5.0 | 225 |
| 5 | 1 | 1 | 1 | 10 | 0 | 0 | 65 | 0 | 5 | 5.0 | 225 |
| 6 | 1 | 1 | 1 | 10 | 0 | 0 | 0 | 0 | 5 | 5.0 | 90 |

| | |
|---|---|
| 7 | Media (Negative Control) |
| 8 | Triton X (Cell Viability/Cytotoxicity Positive Control) |

A change in tonicifier has no significant effect upon PYY₃₋₃₆ in vitro performance. In addition, a change in pH has no impact upon PYY₃₋₃₆ in vitro performance,

### EXAMPLE 10

### Effect of Changing Osmolarity Upon PYY₃₋₃₆ In Vitro Performance

Formulations were manufactured as listed in Table 9. The formulations were then filled into 1-cc amber glass non-silanized vials, 0.5 mL fill per vial, and were fitted with a trifoil-lined polypropylene cap. All in vitro tissue model tested was conducted as described in Example 2.

TABLE 9
Formulations Evaluated in Example 10.

| | | | | | Citrate | | | | | |
|---|---|---|---|---|---|---|---|---|---|---|
| Group | PYY₃₋₃₆ | MBCD | DDPC | EDTA | Buffer | Lactose | Sorbitol | NaCl | | |
| # | (mg/mL) | (mg/mL) | (mg/mL) | (mg/mL) | (mM) | (mM) | (mm) | (mM) | pH | Osmolarity |
| 1 | 1 | 45 | 1 | 1 | 10 | 25 | 100 | 0 | 4.5 | 225 |
| 2 | 1 | 45 | 1 | 1 | 10 | 25 | 100 | 0 | 4.5 | 275 |

| | |
|---|---|
| 3 | Media (Negative Control) |
| 4 | Triton X (Cell Viability and Cytotoxicity Positive Control) |

Osmolarity has a significant impact upon the in vitro performance of PYY₃₋₃₆. In general, it seems that formulations of higher osmolarity (i.e., 275 mOsm/kg) demonstrate a lower permeation than those of a lower osmolarity (i.e., 225 mgOsm/kg).

### EXAMPLE 11

### Presence of Me-b-CD Decreases Preservative Effectiveness of Chlorobutanal

In this example, it was desired to examine the preservative effectiveness of various PYY₃₋₃₆ formulations. The formulations tested are described in Table 10.

TABLE 10
Formulations tested in Example 11.

| Sample | Composition | Testing |
|---|---|---|
| 1 | 1 mg/mL PYY, 45 mg/mL M-b-CD, 1 mg/mL | Modified |
| | EDTA, 1 mg/mL DDPC, 10 mM citrate | PET, pH |
| | (pH 4.3), 25 mM lactose, 100 mM | Appearance |
| | sorbitol, 0.5% CB | |
| 2 | 45 mg/mL M-b-CD, 1 mg/mL EDTA, 1 mg/mL | Modified |
| | DDPC, 10 mM citrate (pH 4.3), 25 mM | PET, pH |
| | lactose, 100 mM sorbitol, 0.5% CB | Appearance |
| 3 | 45 mg/mL M-b-CD, 1 mg/mL EDTA, 1 mg/mL | Modified |
| (30 mL) | DDPC, 10 mM citrate (pH 4.3), 25 mM | PET, pH |
| | lactose, 100 mM sorbitol | Appearance |
| 4 | 10 mM citrate (pH 4.3), 145 mM NaCl, | Modified |
| (30 mL) | 0.5% CB | PET, pH |
| | | Appearance |
| 5 | 45 mg/mL M-b-CD, 1 mg/mL EDTA, 10 mM | Modified |
| (30 mL) | citrate (pH 4.3), 25 mM lactose, 100 | PET, pH |
| | mM sorbitol, 0.5% CB | Appearance |
| 6 | 45 mg/mL M-b-CD, 1 mg/mL EDTA, | Modified |
| (30 mL) | 1 mg/mL DDPC, 10 mM citrate (pH 4.3), | PET, pH |
| | 0.5% CB | Appearance |
| 7 | 1 mg/mL EDTA, 1 mg/mL DDPC, 10 mM | Modified |
| (30 mL) | citrate (pH 4.3), 25 mM lactose, 100 | PET, pH |
| | mM sorbitol, 0.5% CB | Appearance |

USP and EP Preservative Effectiveness Testing (PET)

Testing procedures is specified in the USP and EP (US and European Pharmacopeia). Nasal formulations considered “Topical” preparations for USP (Cat. 2), EP (Cat. A).

Table 11 lists the data for preservative effectiveness for the various formulations. Specifically, data are presented for log reduction in various microorganisms for various periods of time. To pass EP requirement, it is necessary that there be at least a 2 log reduction for yeast and mold (C. albicans and A. niger) species at day 14. The data show that was only achieved for the case of samples #4 and #7. IN both cases of #4 and #7, Me-b-CD was absent. These data strongly suggest that presence of Me-b-CD inhibits chlorobutanol's preservative effectiveness.

TABLE 11
Preserative Effectivness of Various Samples Tested in Example 11.
PET Data shows the following results

| | | | | | | | | | | | Good chance in |
|---|---|---|---|---|---|---|---|---|---|---|---|
| Sample | P. aeruginosa | | E. coli | | S. aureus | | C. albicans | | A. niger | | passing USP |
| # | 14 | 28 | 14 | 28 | 14 | 28 | 14 | 28 | 14 | 28 | and EP? |
| 1 | 5.4 | | 5.7 | | 5.6 | | 3.6 | | 1.2 | | USP only |
| 2 | 5.4 | | 5.7 | | 5.6 | | 1.3 | | 1.3 | | USP only |
| 3 | 5.4 | | 5.7 | | 5.6 | | 0.3 | | 1.1 | | USP only |
| 4 | 5.4 | | 5.7 | | 5.6 | | 6.0 | | 2.6 | | Both |
| 5 | 5.4 | | 5.7 | | 5.6 | | 0.9 | | 1.1 | | USP only |
| 6 | 5.4 | | 5.7 | | 5.6 | | 1.2 | | 1.0 | | USP only |
| 7 | 5.4 | | 5.7 | | 5.6 | | 6.0 | | 3.8 | | Both |

### EXAMPLE 12

### Effect of Sodium Benzoate Level on Preserative Effectiveness of PYY₃₋₃₆ when Formulated in the Presence of Me-b-CD

The following Table 12 lists the formulations evaluated:

TABLE 12
Samples Tested in Example 12.

| Sample | Composition | Comments |
|---|---|---|
| 1 | 45 mg/mL M-b-CD, 1 mg/mL EDTA, | Current lead |
| (30 mL) | 1 mg/mL DDPC, 10 mM citrate (pH 4.3), | formulation |
| | 25 mM lactose, 100 mM sorbitol, | w/o CB w/0.5% |
| | 0.50% Na Benzoate | Na Benzoate; |
| | | pH 4.3 |
| 2 | 45 mg/mL M-b-CD, 1 mg/mL EDTA, | Current lead |
| (30 mL) | 1 mg/mL DDPC, 10 mM citrate (pH 4.3), | formulation |
| | 25 mM lactose, 100 mM sorbitol, | w/o CB w/0.25% |
| | 0.25% Na Benzoate | Na Benzoate; |
| | | pH 4.3 |
| 3 | 45 mg/mL M-b-CD, 1 mg/mL EDTA, | Current lead |
| (30 mL) | 1 mg/mL DDPC, 10 mM citrate (pH 4.3), | formulation |
| | 25 mM lactose, 100 mM sorbitol, | w/o CB w/0.1% |
| | 0.10% Na Benzoate | Na Benzoate; |
| | | pH 4.3 |
| 4 | 45 mg/mL M-b-CD, 1 mg/mL EDTA, | Current lead |
| (30 mL) | 1 mg/mL DDPC, 10 mM citrate (pH 4.3), | formulation |
| | 25 mM lactose, 100 mM sorbitol, | w/o CB w/0.05% |
| | 0.05% Na Benzoate | Na Benzoate; |
| | | pH 4.3 |

USP and EP Preservative Effectiveness Testing (PET)

Testing procedures is specified in the USP and EP (US and European Pharmacopeia). Nasal formulations considered “Topical” preparations for USP (Cat. 2), EP (Cat. A).

Table 13 lists the data for preservative effectiveness for the various formulations. Specifically, data are presented for log reduction in various microorganisms for various periods of time. To pass EP requirement, it is necessary that there be at least a 2 log reduction for yeast and mold (C. albicans and A. niger) species at day 14. The data show that this level was achieved when the level of sodium benzoate was 0.5%.

TABLE 13
Preserative Effectivness of Various Samples Tested in Example 11.

| | | | | | | | | | | | | Good chance |
|---|---|---|---|---|---|---|---|---|---|---|---|---|
| | | P. aeruginosa | | E. coli | | S. aureus | | C. albicans | | A. niger | | for Passeing |
| # | Sample | 14 | 28 | 14 | 28 | 14 | 28 | 14 | 28 | 14 | 28 | USP and EP? |
| 1 | w/o CB, w/0.5% | 5.2 | | 5.2 | | 5.5 | | 5.5 | | 5.4 | | Both |
| | Na Benzoate | | | | | | | | | | | |
| 2 | w/o CB, w/0.25% | 5.2 | | 5.2 | | 5.5 | | 1.5 | | 2.1 | | USP only |
| | Na Benzoate | | | | | | | | | | | |
| 3 | w/o CB, w/0.1% | 5.2 | | 5.2 | | 5.5 | | 0.6 | | 0.8 | | USP only |
| | Na Benzoate | | | | | | | | | | | |
| 4 | w/o CB, w/0.05% | 5.2 | | 5.2 | | 5.5 | | 1.1 | | 1.1 | | USP only |
| | Na Benzoate | | | | | | | | | | | |

### EXAMPLE 13

### Effect of Various Buffers on Stability Towards Thermal Stress for PYY₃₋₃₆ in the Absence of any Additional Excipients

Formulations were manufactured as outlined in Table 14. The buffers tested were citrate, tartarate, acetate, and glutamate. In all cases, PYY₃₋₃₆ was present at 1 mg/mL and the pH was 5.0. These formulations were then filled into 1-cc amber non-silanized vials, 1 mL fill per vial, and fitted with a trifoil-lined polypropylene cap. Samples were stored and tested for recovery as described in Example 3 except that additional temperatures were evaluated, specifically, 50° C., 40° C., and 25° C.

TABLE 14
Formulations evaluated in Example 13.

| | | Citrate | Tartarate | Acetate | Glutamate | |
|---|---|---|---|---|---|---|
| Group | PYY₃₋₃₆ | Buffer | Buffer | Buffer | Buffer | |
| # | (mg/mL) | (mM) | (mM) | (mM) | (mM) | pH |
| 1 | 1 | 10 | | | | 5.0 |
| 2 | 1 | | 10 | | | 5.0 |
| 3 | 1 | | | 10 | | 5.0 |
| 4 | 1 | | | | 10 | 5.0 |

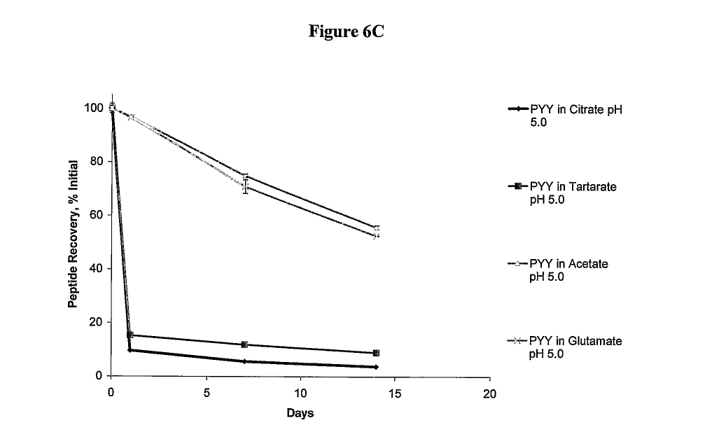

The HPLC data show that the best stability (highest recovery after storage at the various conditions) were the acetate and glutamate buffers. The peptide recovery results are shown in FIGS. 6A, 6B and 6C for 25, 40 and 50 C, respectively.

The results of this experiment are illuminating in that those buffers that best preserved PYY₃₋₃₆ stability were monovalent buffers, whereas those that did not improve PYY₃₋₃₆ stability were polyvalent buffers. Monovalent buffers increase PYY₃₋₃₆ stability under thermal stress.

### EXAMPLE 14

### Effect of Buffer Type and pH on Stability Under Thermal Stress of PYY₃₋₃₆ in the Presence of Sorbitol as Tonicifier

Formulations were manufactured as outlined in Table 15. The buffers tested were citrate, acetate, and glutamate. In all cases, PYY₃₋₃₆ was present at 2 mg/mL and sorbitol was present to provide osmolarity of 225 mOsm. The pH was varied from 3.5 to 5.0. These formulations were then filled into 1-cc amber non-silanized vials, 1 mL fill per vial, and fitted with a trifoil-lined polypropylene cap. Samples were stored and tested for recovery as described in Example 3.

TABLE 15
Formulations evaluated in Example 14.

| | | Citrate | Acetate | Glutamate | | |
|---|---|---|---|---|---|---|
| Group | PYY | Buffer | Buffer | Buffer | Sorbitol | |
| # | (mg/mL) | (mM) | (mM) | (mM) | (mM) | pH |
| 1 | 2 | 10 | 0 | 0 | 210 | 5.0 |
| 2 | 2 | 10 | 0 | 0 | 210 | 4.5 |
| 3 | 2 | 10 | 0 | 0 | 210 | 4.0 |
| 4 | 2 | 10 | 0 | 0 | 210 | 3.5 |
| 5 | 2 | 0 | 10 | 0 | 210 | 5.0 |
| 6 | 2 | 0 | 10 | 0 | 210 | 4.5 |
| 7 | 2 | 0 | 10 | 0 | 210 | 4.0 |
| 8 | 2 | 0 | 10 | 0 | 210 | 3.5 |
| 9 | 2 | 0 | 0 | 10 | 210 | 5.0 |
| 10 | 2 | 0 | 0 | 10 | 210 | 4.5 |
| 11 | 2 | 0 | 0 | 10 | 210 | 4.0 |
| 12 | 2 | 0 | 0 | 10 | 210 | 3.5 |
| 13 | 2 | 0 | 0 | 0 | 0 | 5.0 |
| 14 | 2 | 0 | 0 | 0 | 0 | 4.5 |
| 15 | 2 | 0 | 0 | 0 | 0 | 4.0 |
| 16 | 2 | 0 | 0 | 0 | 0 | 3.5 |

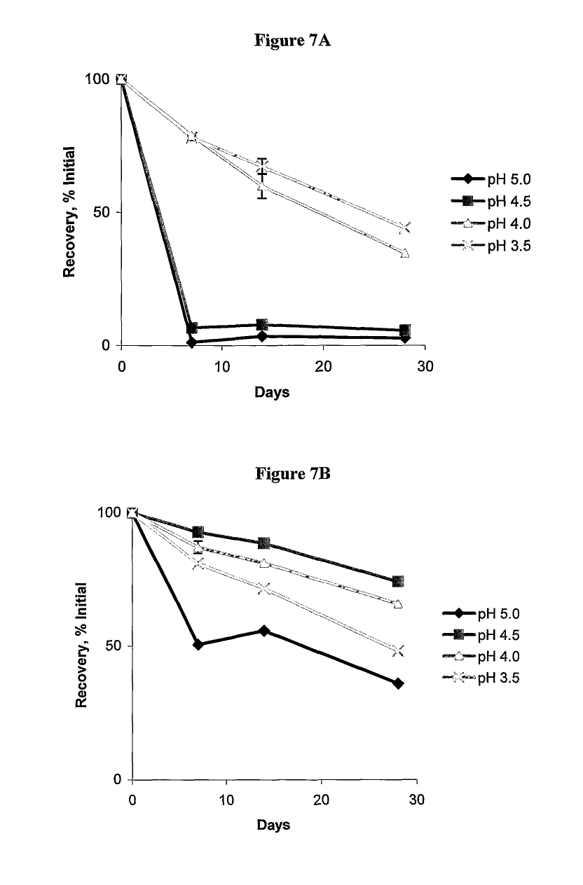

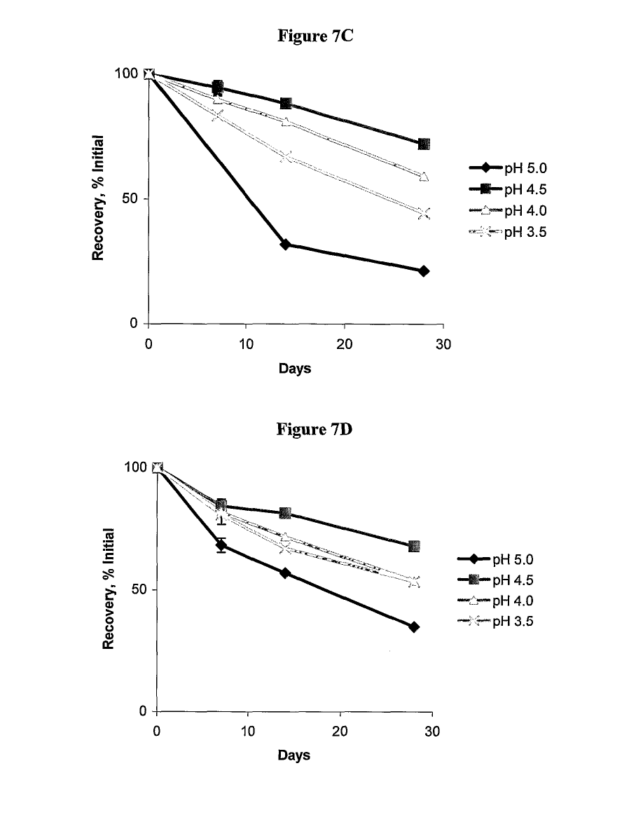

The HPLC data show that the best performers over the temperatures evaluated are the acetate and glutamate buffers as well as the unbuffered formulations. The peptide recovery results are depicted in FIGS. 7A, 7B, 7C and 7D for cases where the buffer was citrate, acetate, glutamate or no buffer, respectively.

As in Example 13, the best-performing formulations are those that contain either a monovalent buffer (i.e., acetate or glutamate) or that do not contain a buffer over the pH range and temperatures evaluated. Those formulations containing a polyvalent buffer (i.e., citrate) did not reach optimal performance. In addition, it appears that optimal stability-maintaining pH for PYY₃₋₃₆ appears to be pH 4.0-pH 5.0 regardless of buffer used.

### EXAMPLE 14

### Effect of Buffer Type and pH on Stability Toward Thermal and Atomization Stresses for PYY₃₋₃₆ in the Presence of Methyl-β-Cylcodextrin, Edetate Disodium, L-α-phosphatidylcholine Didecanoyl, Lactose, and Sorbitol

Formulations were manufactured as outlined in Table 16. The buffers tested were citrate, acetate, and glutamate. In all cases, PYY₃₋₃₆ was present at 6 mg/mL, Me-b-CD present at 45 mg/mL, EDTA present at 1 mg/mL, DDPC present at 1 mg/mL, lactose present at 25 mM, sorbitol present at 100 mM, and chlorobutanol present at 5 mg/mL. sorbitol was present to provide osmolarity of 225 mOsm. The pH was varied from 4.0 to 5.0.

These formulations were filled into 3-cc amber non-silanized vials, 3.9 mL fill per vial, and fitted with a 100 μL actuator. Actuators were manually primed by actuation four times. Each vial was stored 30° C./65% RH and were actuated thrice daily with a minimum of one hour between actuations. The actuation stress continued for a total of ten days. The stability-indicating HPLC method used is outlined in Example 3.

TABLE 16
Formulations evaluated in Example 14.

| | | | | | Citrate | Acetate | Glutatmate | | | | |
|---|---|---|---|---|---|---|---|---|---|---|---|
| Group | PYY | MβCD | EDTA | DDPC | Buffer | Buffer | Buffer | Lactose | Sorbitol | CB | |
| # | (mg/mL) | (mg/mL) | (mg/mL) | (mg/mL) | (mM) | (mM) | (mM) | (mM) | (mM) | (mg/mL) | pH |
| 1 | 6 | 45 | 1 | 1 | 10 | | | 25 | 100 | 5 | 5.0 |
| 2 | 6 | 45 | 1 | 1 | 10 | | | 25 | 100 | 5 | 4.6 |
| 3 | 6 | 45 | 1 | 1 | 10 | | | 25 | 100 | 5 | 4.3 |
| 4 | 6 | 45 | 1 | 1 | 10 | | | 25 | 100 | 5 | 4.0 |
| 5 | 6 | 45 | 1 | 1 | | 10 | | 25 | 100 | 5 | 5.0 |
| 6 | 6 | 45 | 1 | 1 | | 10 | | 25 | 100 | 5 | 4.6 |
| 7 | 6 | 45 | 1 | 1 | | 10 | | 25 | 100 | 5 | 4.3 |
| 8 | 6 | 45 | 1 | 1 | | 10 | | 25 | 100 | 5 | 4.0 |
| 9 | 6 | 45 | 1 | 1 | | | 10 | 25 | 100 | 5 | 5.0 |
| 10 | 6 | 45 | 1 | 1 | | | 10 | 25 | 100 | 5 | 4.6 |
| 11 | 6 | 45 | 1 | 1 | | | 10 | 25 | 100 | 5 | 4.3 |
| 12 | 6 | 45 | 1 | 1 | | | 10 | 25 | 100 | 5 | 4.0 |
| 13 | 6 | 45 | 1 | 1 | | | | 25 | 100 | 5 | 5.0 |
| 14 | 6 | 45 | 1 | 1 | | | | 25 | 100 | 5 | 4.6 |
| 15 | 6 | 45 | 1 | 1 | | | | 25 | 100 | 5 | 4.3 |
| 16 | 6 | 45 | 1 | 1 | | | | 25 | 100 | 5 | 4.0 |

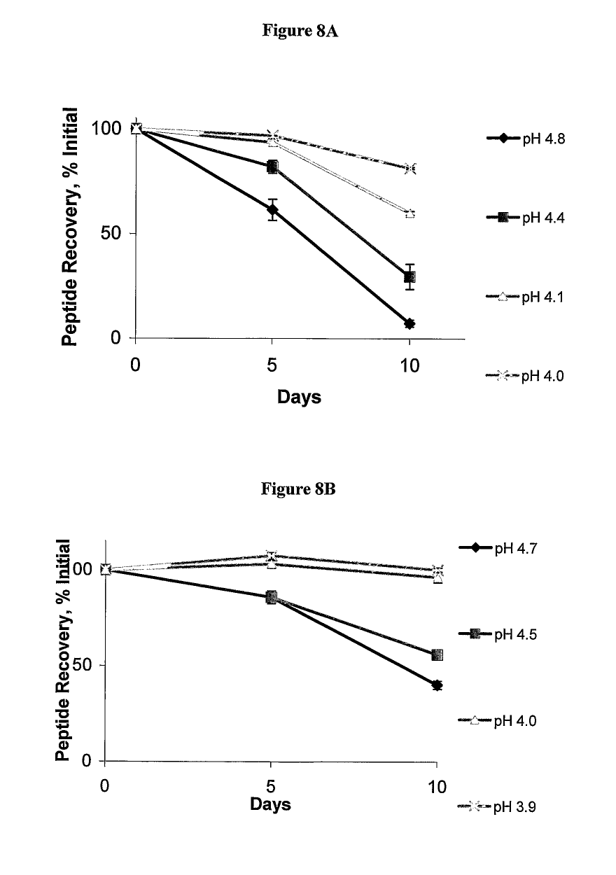

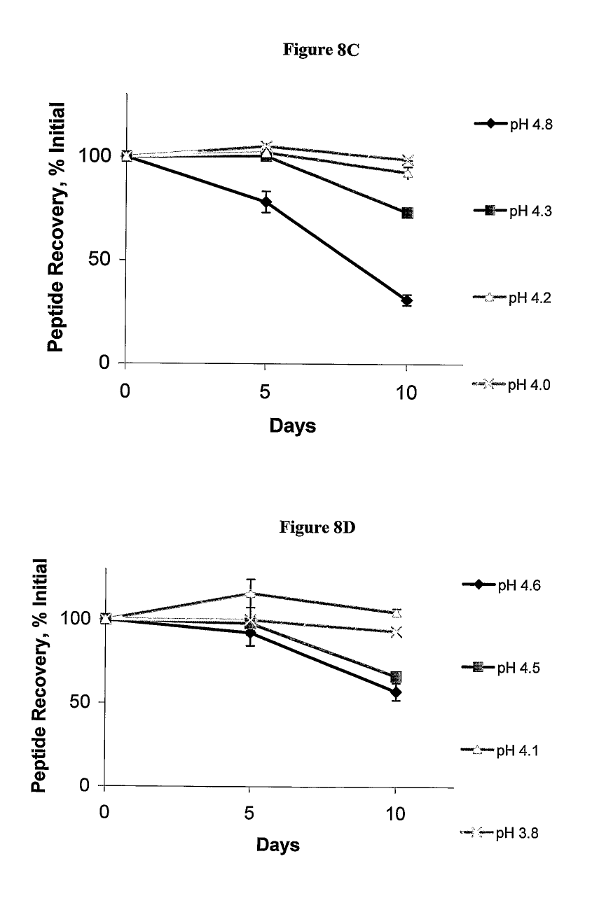

The HPLC data show that the best performers over the temperatures evaluated are the acetate and glutamate buffers as well as the unbuffered formulations. The peptide recovery results are tabulated in FIGS. 8A, 8B, 8C and 8D for the cases of citrate, acetate, glutamate and no buffer, respectively. Figures illustrating the structures of the buffers used in this study can be found in Example 10.

The best-performing formulations are those that contain either a monovalent buffer (i.e., acetate or glutamate) or that do not contain a buffer over the pH range and temperatures evaluated. Those formulations containing a polyvalent buffer (i.e., citrate) did not reach optimal performance. In addition, it appears that optimal stability-maintaining pH for PYY₃₋₃₆ appears to be pH 4.0-pH 5.0 regardless of buffer used.

What is claimed is:

1. An aqueous Y2 receptor-binding peptide formulation suitable for transmucosal administration, comprising a Y2 receptor-binding peptide, a cyclodextrin and an effective amount of an anti-microbial preservative, wherein said Y2 receptor-binding peptide is PYY, PYY (3-36) or an analogue of PYY or an analog of PYY (3-36).

2. The Y2 receptor-binding peptide formulation of claim 1, wherein the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin.

3. The Y2 receptor-binding peptide formulation of claim 2, wherein the cyclodextrin is methyl-β-cyclodextrin.

4. The Y2 receptor-binding peptide formulation of claim 1, wherein the preservative is sodium benzoate.

5. The Y2 receptor-binding peptide formulation of claim 4, wherein the formulation has a sodium benzoate concentration greater than about 0.05%.

6. The Y2 receptor-binding peptide formulation of claim 4, wherein the formulation has a sodium benzoate concentration greater than about 0.10%.

7. The Y2 receptor-binding peptide formulation of claim 4, wherein the formulation has a sodium benzoate concentration greater than about 0.25%.

8. The Y2 receptor-binding peptide formulation of claim 4, wherein the formulation has a sodium benzoate concentration greater than about 0.50%.

9. The Y2 receptor-binding peptide formulation of claim 4, wherein the formulation has a pH between 3.0 and 5.0.

10. The Y2 receptor-binding peptide formulation of claim 9, wherein the formulation has a pH between 3.5 and 4.5.

11. The Y2 receptor-binding peptide formulation of claim 2, wherein the preservative-effectiveness meets United State Pharmacopoeia for Category 2 criteria.

12. The Y2 receptor-binding peptide formulation of claim 2, wherein the preservative-effectiveness meets European Pharmacopoeia Category A criteria.

13. The Y2 receptor-binding peptide formulation of claim 2, wherein the preservative-effectiveness meets European Pharmacopoeia Category B criteria.

14. The Y2 receptor-binding peptide formulation of claim 2, wherein the preservative effectively causes at least a 2 log reduction for a yeast and a mold at day 14.

15. The Y2 receptor-binding peptide formulation of claim 14, wherein the preservative effectively causes at least a 2 log reduction for C. albicans and A. niger species.

16. The Y2 receptor-binding peptide formulation of claim 2, wherein the preservative effectively causes at least a 1 log reduction for a yeast and a mold at day 14.

17. The Y2 receptor-binding peptide formulation of claim 16, wherein the preservative effectively causes at least a 1 log reduction for C. albicans and A. niger species.

18. The Y2 receptor-binding peptide formulation of claim 1, wherein the Y2 receptor-binding peptide is a PYY peptide comprised of an amino acid sequence selected from the group consisting of SEQ ID NOs: 1-21, SEQ ID NOs: 72, 73 and 74, and SEQ ID NOs: 90-105.

19. The Y2 receptor-binding peptide formulation of claim 19, wherein the PYY peptide is a PYY(3-36) peptide.

20. The Y2 receptor-binding peptide formulation of claim 20, wherein the PYY(3-36) peptide is comprised of an amino acid sequence selected from the group consisting of SEQ ID NOs: 2, 3 and SEQ ID NOs: 90-105.

21. An aqueous Y2 receptor-binding peptide formulation suitable for transmucosal administration, comprising a Y2 receptor-binding peptide and a cyclodextrin, wherein such formulation has been prepared aseptically, and wherein said Y2 receptor-binding peptide is PYY, PYY (3-36) or an analogue of PYY or an analog of PYY (3-36).

22. The Y2 receptor-binding peptide formulation of claim 22, wherein the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin.

23. The Y2 receptor-binding peptide formulation of claim 23, wherein the cyclodextrin is methyl-β-cyclodextrin.

24. A use of an Y2 receptor-binding peptide in the manufacture of a medicament for treating obesity eating in a human, comprised of intranasally administering to said human a therapeutically effective amount of an aqueous formulation, wherein the formulation comprises said Y2 receptor-binding peptide, a cyclodextrin and sodium benzoate, and wherein said Y2 receptor-binding peptide is PYY, PYY (3-36) or an analogue of PYY or an analog of PYY (3-36).

25. The use of an Y2 receptor-binding peptide of claim 25, to induce weight-loss or satiety.

26. The use of an Y2 receptor-binding peptide of claim 25, wherein the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin.

27. The use of an Y2 receptor-binding peptide of claim 27, wherein the cyclodextrin is methyl-β-cyclodextrin.

28. The use of an Y2 receptor-binding peptide of claim 25, wherein the preservative is sodium benzoate.

29. The use of an Y2 receptor-binding peptide of claim 29, wherein the formulation has a sodium benzoate concentration greater than about 0.25%.

30. The use of an Y2 receptor-binding peptide of claim 29, wherein the formulation has a sodium benzoate concentration greater than about 0.50%.

31. The use of an Y2 receptor-binding peptide of claim 29, wherein the formulation has a pH between 3.0 and 5.0.

32. The use of an Y2 receptor-binding peptide of claim 29, wherein the formulation has a pH between 3.5 and 4.5.

33. The use of an Y2 receptor-binding peptide of claim 25, wherein the Y2 receptor-binding peptide is PYY or PYY (3-36) or an analogue of PYY or an analog of PYY (3-36).

34. The use of an Y2 receptor-binding peptide of claim 25, wherein the Y2 receptor-binding peptide is a PYY peptide comprised of an amino acid sequence selected from the group consisting of SEQ ID NOs: 1-21, SEQ ID NOs: 72, 73 and 74, and SEQ ID NOs: 90-105.

35. The use of an Y2 receptor-binding peptide of claim 35, wherein the PYY peptide is a PYY(3-36) peptide.

36. The use of an Y2 receptor-binding peptide of claim 36, wherein the PYY(3-36) peptide is comprised of an amino acid sequence selected from the group consisting of SEQ ID NOs: 2, 3 and SEQ ID NOs: 90-105.

37. A use of an Y2 receptor-binding peptide in the manufacture of a medicament for treating obesity eating in a human, comprised of intranasally administering to said human a therapeutically effective amount of an aqueous formulation, wherein the formulation comprises said Y2 receptor-binding peptide and a cyclodextrin, and wherein such formulation has been prepared aseptically, and wherein said Y2 receptor-binding peptide is PYY, PYY (3-36) or an analogue of PYY or an analog of PYY (3-36).

38. The use of an Y2 receptor-binding peptide of claim 38, wherein the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin.

39. The use of an Y2 receptor-binding peptide of claim 39, wherein the cyclodextrin is methyl-β-cyclodextrin.

40. An aqueous Y2 receptor-binding peptide formulation suitable for transmucosal administration, comprising a Y2 receptor-binding peptide, a cyclodextrin, and having a pH between 3.0 and 6.0.

41. The Y2 receptor-binding peptide formulation of claim 41, wherein the osmolarity of the formulation is between about 50 and about 300 mOsm.

42. The Y2 receptor-binding peptide formulation of claim 41, wherein the osmolarity of the formulation is between about 150 and about 275 mOsm.

43. The Y2 receptor-binding peptide formulation of claim 41, wherein the osmolarity of the formulation is between about 180 and about 260 mOsm.

44. The Y2 receptor-binding peptide formulation of claim 42, wherein the formulation is free of sugars.

45. The Y2 receptor-binding peptide formulation of claim 45, wherein the osmolarity of the formulation is adjusted using an inorganic salt.

46. The Y2 receptor-binding peptide formulation of claim 46, wherein the inorganic salt is sodium chloride.

47. The Y2 receptor-binding peptide formulation of claim 41, wherein the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin.

48. The Y2 receptor-binding peptide formulation of claim 46, wherein the cyclodextrin is methyl-β-cyclodextrin.

49. The Y2 receptor-binding peptide formulation of claim 41, wherein the pH of the formulation is between about 3.5 and about 5.5.

50. The Y2 receptor-binding peptide formulation of claim 41, wherein the pH of the formulation is between about 3.5 and about 5.0.

51. The Y2 receptor-binding peptide formulation of claim 41, wherein the pH of the formulation is between about 3.7 and about 4.7.

52. The Y2 receptor-binding peptide formulation of claim 41, wherein the pH of the formulation is controlled by a buffer salt, and said buffer salt has a net single ionogenic moiety with a pKₐ within two pH units of the pH of the formulation.

53. The Y2 receptor-binding peptide formulation of claim 41, wherein said buffer salt has a net single ionogenic moiety with a pKₐ within one pH unit of the pH of the formulation.

54. The Y2 receptor-binding peptide formulation of claim 53, wherein said buffer salt is selected from the list consisting of glutamate, acetate, glycine, histidine, arginine, lysine, methionine, lactate, formate, and glycolate.

55. The Y2 receptor-binding peptide formulation of claim 41, wherein the Y2 receptor-binding peptide is PYY or an analogue of PYY.

56. The Y2 receptor-binding peptide formulation of claim 41, wherein the Y2 receptor-binding peptide is a PYY peptide comprised of an amino acid sequence selected from the group consisting of SEQ ID NOs: 1-21, SEQ ID NOs: 72, 73 and 74, and SEQ ID NOs: 90-105.

57. The Y2 receptor-binding peptide formulation of claim 57, wherein the PYY peptide is a PYY(3-36) peptide.

58. The Y2 receptor-binding peptide formulation of claim 58, wherein the PYY(3-36) peptide is comprised of an amino acid sequence selected from the group consisting of SEQ ID NOs: 2, 3 and SEQ ID NOs: 90-105.

59. A use of an Y2 receptor-binding peptide in the manufacture of a medicament for treating obesity eating in a mammal, comprising intranasally administering to said mammal a therapeutically effective amount of an aqueous formulation comprised said Y2 receptor-binding peptide wherein the formulation is comprised of a cyclodextrin, has a pH between 3.0 and 6.0 and an osmolarity between about 50 mOsm and about 300 mOsm.

60. The use of an Y2 receptor-binding peptide of claim 60, to induce weight-loss or to induce satiety.

61. The use of an Y2 receptor-binding peptide of claim 60, wherein the osmolarity of the formulation is between about 150 and about 275 mOsm.

62. The use of an Y2 receptor-binding peptide of claim 60, wherein the osmolarity of the formulation is between about 180 and about 260 mOsm.

63. The use of an Y2 receptor-binding peptide of claim 60, wherein the formulation is free of sugars.

64. The use of an Y2 receptor-binding peptide of claim 60, wherein the osmolarity of the formulation is adjusted using an inorganic salt.

65. The use of an Y2 receptor-binding peptide of claim 60, wherein the inorganic salt is sodium chloride.

66. The use of an Y2 receptor-binding peptide of claim 60, the cyclodextrin is selected from the group consisting of α-cyclodextrin, β-cyclodextrin, γ-cyclodextrin, a β-cyclodextrin derivatives, 2-hydroxypropyl-β-cyclodextrin, a methylated cyclodextrin, methyl-β-cyclodextrin, dimethyl-β-cyclodextrin, an ethylated cyclodextrin, a hydroxypropylated cyclodextrin and a polymeric cyclodextrin.

67. The use of an Y2 receptor-binding peptide of claim 67, wherein the cyclodextrin is methyl-β-cyclodextrin.

68. The use of an Y2 receptor-binding peptide of claim 60, wherein the pH of the formulation is between about 3.5 and about 5.5.

69. The use of an Y2 receptor-binding peptide of claim 60, wherein the pH of the formulation is between about 3.5 and about 5.0.

70. The use of an Y2 receptor-binding peptide of claim 60, wherein the pH of the formulation is between about 3.7 and about 4.7.

71. The use of an Y2 receptor-binding peptide of claim 60, wherein the pH of the formulation is controlled by a buffer salt, and said buffer salt has a net single ionogenic moiety with a pKₐ within two pH units of the pH of the formulation.

72. The use of an Y2 receptor-binding peptide of claim 41, wherein said buffer salt has a net single ionogenic moiety with a pKₐ within one pH unit of the pH of the formulation.

73. The use of an Y2 receptor-binding peptide of claim 72, wherein said buffer is selected from the list consisting of glutamate, acetate, glycine, histidine, arginine, lysine, methionine, lactate, formate, and glycolate.

74. The use of an Y2 receptor-binding peptide of claim 60, wherein the Y2 receptor-binding peptide is PYY or PYY (3-36) or an analogue of PYY or an analog of PYY (3-36).

75. The use of an Y2 receptor-binding peptide of claim 60, wherein the Y2 receptor-binding peptide is a PYY peptide comprised of an amino acid sequence selected from the group consisting of SEQ ID NOs: 1-21, SEQ ID NOs: 72, 73 and 74, and SEQ ID NOs: 90-105.

76. The use of an Y2 receptor-binding peptide of claim 76, wherein the PYY peptide is a PYY(3-36) peptide.

77. The use of an Y2 receptor-binding peptide of claim 77, wherein the PYY(3-36) peptide is comprised of an amino acid sequence selected from the group consisting of SEQ ID NOs: 2, 3 and SEQ ID NOs: 90-105.

78. An aqueous Y2 receptor-binding peptide formulation suitable for transmucosal administration, comprising said Y2 receptor-binding peptide, a cyclodextrin and has a pH between 3.0 and 6.0, wherein said Y2 receptor-binding peptide is PYY, PYY (3-36) or an analogue of PYY or an analog of PYY (3-36).

79. A use of an Y2 receptor-binding peptide in the manufacture of a medicament for treating obesity eating in a mammal, comprising intranasally administering to said mammal a therapeutically effective amount of an aqueous formulation comprised said Y2 receptor-binding peptide, wherein the formulation is comprised of a cyclodextrin and has a pH between 3.0 and 6.0 and an osmolarity between about 50 mOsm and about 300 mOsm, and wherein said Y2 receptor-binding peptide is PYY, PYY (3-36) or an analogue of PYY or an analog of PYY (3-36).
